(19) United States
(12) Patent Application Publication
(10) Pub. No.: US 2022/0403469 A1
(43) Pub. Date: Dec. 22, 2022

(54) Precision Medicine for Schizophrenia and Psychotic Disorders: Objective Assessment, Risk Prediction, Pharmacogenomics, and Repurposed Drugs

(71) Applicant: Indiana University Research and Technology Corporation, Indianapolis, IN (US)

(72) Inventor: Alexander Bogdan Niculescu, III, Indianapolis, IN (US)

(73) Assignee: United States Government as Represented by the Department of Veterans Affairs, Washington, DC (US)

(21) Appl. No.: 17/351,132

(22) Filed: Jun. 17, 2021

(51) Int. Cl. C12Q 1/6883; G16B 25/10

(52) U.S. Cl. CPC C12Q 1/6883; G16B 25/10; C12Q 2600/158

(57) ABSTRACT

Disclosed are novel compounds for treating and preventing schizophrenia, and more generally psychosis, by bioinformatics drug repurposing using novel genes expression biomarkers involved in psychotic symptoms (delusions, hallucinations); methods for assessing severity, determining future risk, matching with a drug treatment, and measuring response to treatment, for psychosis in a subject; and method of using repurposed drugs and natural compounds to prevent and to treat psychosis. Methods are disclosed using a universal approach, in everybody, as well as personalized approaches by gender. The discovery describes compounds for use in everybody (universal), as well as personalized by gender (males, females). Methods for identifying which subjects should be receiving which treatment, using genes expression biomarkers for patient stratification and measuring response to treatment. The disclosure also relates to algorithms. The algorithms combine biomarkers as well as clinical measures for psychosis, to identify subjects who are at risk of psychosis, and to track responses to treatments.

### STATEMENT OF GOVERNMENT SUPPORT

This invention was made with government support under OD007363 awarded by the National Institutes of Health and 2IO1CX000139 merit award by the Veterans Administration. The government has certain rights in the invention.

### BACKGROUND

Schizophrenia is a heterogeneous disorder, composed of positive and negative psychotic symptoms. Psychotic symptoms, more broadly, are also often present in other psychiatric disorders. They can be difficult to assess, as they are based on the patient's self report and on the clinician's clinical impression. Continued improvements are needed to adequately diagnose and treat individuals suffering psychotic symptoms.

### SUMMARY

Provided here are newly identified blood gene expression biomarkers for hallucinations, and for delusions. The biomarkers provide a means of assessing state severity, short-term risk, and long-term risk. The biomarkers can also be used for drug repurposing.

Some aspects of the invention include methods for treating an individual experiencing or at a heighted risk for developing symptoms such as delusions and/or hallucinations. These symptoms may be indicative of certain psychiatric disorders such as psychosis and/or schizophrenia. Treating involves administered at least one course of treatment, treatment may include psychiatric counseling, administering certain physical intervention, and/or prescribing and/or administering at least one therapeutic compound. Treating may include at least one of the following outcomes, curing, mitigating, managing or otherwise recuing the severity and/or the number of frequency delusions and or hallucinations. In some aspects of the invention treating may include identifying individuals with or at an increased risk for developing delusions and/or hallucinations my measuring the level of certain RNA biomarkers as identified in, for example, Tables 2 and 3.

Some aspects of the invention include methods for diagnosing an individual experiencing or at a heighted risk for developing symptoms such as delusions and/or hallucinations. These symptoms may be indicative of certain psychiatric disorders such as psychosis and/or schizophrenia. Diagnosing does not require treatment, although it may lead to, or become, part of treating an individual who is manifesting or at an increased risk for manifesting symptom of certain types of mental illness such as psychosis and or schizophrenia. In some aspects of the invention diagnosing may include identifying individuals with or at an increased risk for developing delusions and/or hallucinations my measuring the level of certain RNA biomarkers as identified in, for example, Tables 2 and 3.

A first embodiment of the invention is a method for treating at least on psychiatric disorder, for example delusions or an increased for developing delusions in an individual, comprising the steps of: (a) obtaining a biological sample from an individual and quantifying the amounts of RNA biomarkers in the biological sample, to create a panel of RNA biomarkers, (b) quantifying the amounts of the RNA biomarkers in the panel in a clinically relevant population to generate a reference expression level for the RNA biomarkers in a panel of RNA biomarkers; (c) comparing the amounts of the biomarkers in the biological sample from the individual with the amounts of the RNA biomarkers present in the reference standard to generate a score for each biomarker; wherein the biomarkers in the a first panel (a) comprise one or more of the following RNA biomarkers: Activator Of Transcription and Developmental Regulator 2 (AUTS2), Pyruvate Dehyrogenase Phosphatase Catalytic Subunit 1(PDP1), Nuclear Receptor Subfamily 4 Group A Member 2 (NR4A2), GNAS Complex Locus (GNAS), Interleukin 6 Signal Transduce (IL6ST), Chromodomain Helicase DNA Binding Protein 9 (CHD9), X-Ray Repair Cross Complementing 6 (XRCC6), RAR Related Orphan Receptor A (RORA), Actinin Alpha 4 (ACTN4), and Acyl-CoA Synthetase Long Chain Family Member 4 (ACSL4), wherein the expression level of the biomarker(s) in the sample is increased relative to a reference expression level, denoting delusions or an increased risk for developing delusions; and biomarkers in a second panel (b) comprise one or more of the following RNA biomarkers: Zinc Finger And BTB Domain Containing 20 (ZBTB20), Forkhead Box P1 (FOXP1), Spondin 1 (SPON1), and (NRP2), wherein the expression level of the RNA biomarker(s) in the sample is decreased relative to a reference expression level of the RNA biomarkers in the panel, denoting delusions or an increased risk for developing delusions; (d) generating a score for the panel of RNA biomarkers, based on the scores of the biomarker(s) in the panel; (e) determining a reference score for the panel in a clinically normal relevant population; (f) identifying a difference between the score of the panel of biomarker(s) in the sample and the reference score of the panel of biomarker(s); (g) identifying the individual as having delusions or of having an elevated risk for developing delusions, based on the difference between the biomarker panel score of the individual relative to the biomarker panel score of the reference; (h) treating the individual identified as having delusions or an elevated risk of delusions with at least one treatment selected from the group consisting of: a treatment based on clinical practice guidelines, administering a therapeutically effective amount of at least one therapeutic drug wherein the mode of treatment is on the specific biomarkers scores indicating that individual will benefit from a particular therapy, treating includes curing, mitigating, reducing or even eliminating symptoms of psychotic disorders such as schizophrenia. In some embodiments of the invention samples are taken from an individual two or more time in order to treat, diagnose, and/or monitor the presence of at least one psychotic disorder such as schizophrenia. In some embodiments, an individual may be treated for symptoms such as delusions or hallucinations without a formal diagnosis of a specific psychotic disorder. In some embodiments, the individual may be treated with drugs known to treat mental illness and/or drugs repurposed to treat mental illness. Samples from an individual may include any or all of the following, tissue samples, bodily fluids such as blood serum, plasma, saliva, cerebral fluid and the like. The samples may be further processed such as by extraction or purification before being analyzed for the presence of one or more biomarkers of interest.

In a third embodiment an individual exhibiting symptoms of a psychotic disorder such as those noted the first and second embodiments may be treated with at least drug selected from the group consisting of: adenosine phosphate, N-acetyl-L-leucine, eldeline, pempidine, verteporfin, C-75, oxprenolol, Prestwick-675, meglumine, guanethidine, pancuronium bromide, karakoline, 15(S)-15-methylprostaglandin E2, hexylcaine, dicoumarol, apramycin, mephenytoin, estriol, 528116.cdx, Cyclopiazonic Acid, SB 218078, BRD-A36630025, Quinacrine hydrochloride, GF-109203X, BRD-A36630025, N9-isoproplyolomoucine, BMS-536924, BRD-K76951091, BRD-K26304855, trichostatin A, ALW-II-38-3, mitoxantrone, HG-6-64-0, alvocidib, SB-216763, and Caffeic acid phenethyl ester. In some embodiments the drug used to treat the individual may a drug repurposed from another use, see for example the drugs in Table 4, some repurposed drugs may be identified as efficacious for this purpose because their use correlates in a beneficial change in at least one of the Biomarkers listed in Tables 2 and/or 3. A fourth embodiment includes at least portion of the first through the third embodiments, wherein the individual is male, and the biomarkers in a first panel (a) comprise one or more biomarkers selected from the group consisting of: Activator Of Transcription And Developmental Regulator (AUTS2), Pyruvate Dehyrogenase Phosphatase Catalytic Subunit 1 (PDP1), Forkhead Box P1 (FOXP1), GNAS Complex Locus (GNAS), Serine Racemase (SRR), Nuclear Receptor Subfamily 4 Group A Member 2 (NR4A2), X-Ray Repair Cross Complementing 6 (XRCC6), RAR Related Orphan Receptor A (RORA), and Actinin Alpha 4 (ACTN4), wherein the expression level of the biomarker(s) in the sample is increased relative to a reference expression level, denoting increased delusions, or the biomarkers in a second panel (b) comprising one or more biomarkers selected from the group consisting of: Zinc Finger And BTB Domain Containing 20 (ZBTB20), Forkhead Box P1 (FOXP1), Spondin 1 (SPON1), NRP2, wherein the expression level of the biomarker(s) in the sample is decreased relative to a reference expression level, denoting increased delusions. The males identified in this embodiments may be treated with at least one therapeutic drug is selected from the group consisting of: flunisolide, apramycin, adenosine phosphate, guanethidine, 15(S)-15-methylprostaglandin E2, meteneprost, methyldopate, hydralazine, rotenone, phthalylsulfathiazole, N-acetyl-L-leucine, eldeline, tocainide, laudanosine, pempidine, 7-aminocephalosporanic acid, Sulfachlorpyridazine, finasteride, 528116.cdx, SB 218078, Quinacrine hydrochloride, N9-isoproplyolomoucine, ALW-II-38-3, mitoxantrone, HG-6-64-01, Alvocidib, SB-216763, Syk Inhibitor, Cyclopiazonic Acid, GW 441756, LY 225910, AG 82, doxorubicin, mitomycin, and terfenadine.

A fifth embodiment includes at least portion of the first through the third embodiments, wherein the individual is a female and the biomarker is at least one biomarker selected from the group consisting of: Phosphodiesterase 4D Interacting Protein (PDE4DIP), Pyruvate Dehyrogenase Phosphatase Catalytic Subunit 1 (PDP1), Transcription Factor 4 (TCF4), Nuclear Receptor Subfamily 4 Group A Member 2 (NR4A2), Chromodomain Helicase DNA Binding Protein 9 (CHD9), (CLCN3), Activator Of Transcription And Developmental Regulator (AUTS2), and (LDB2), wherein the expression level of the biomarker(s) in the sample is increased relative to a reference expression level, denoting increased delusions; and the biomarkers in a second panel (b) comprise one or more biomarkers selected from the group consisting of (FGFR1), (DISC1), (FGFR2), (SPTBN1), (INSR), (GRIK3), Zinc Finger, and BTB Domain Containing 20 (ZBTB20), wherein the expression level of the biomarker(s) in the sample is decreased relative to a reference expression level, denoting increased delusions. The females identified in this embodiments may be treated with at least one therapeutic drug is selected from the group consisting of: erastin, harpagoside, metacycline, amiodarone, furaltadone, metformin, timolol, Repaglinide, sulfafurazole, PNU-0230031, Probenecid, furosemide, fluphenazine, myricetin, sulfacetamide, lomustine, BCB000039, Harmalol, I-BET151, Nylidrin hydrochloride, AMG 9810, Doxorubicin, Mitomycin C, Fludrocortisone acetate, Purvalanol A, Teniposide, Geldanamycin, Importazole, BRD-A36630025, YM-155, Auranofin, 7643453, G-221, BRD-A49680073, BRD-K08547377, and Cladribine.

A sixth embodiment includes a method of assessing and/or treating schizophrenia and other psychotic disorders in general, and delusions in particular, in an individual, comprising: calculating combined biomarkers and clinical information Up-based on the equation:(Biomarker Panel Score)+(Delusions Score)−(Grooming Score)=Up-Delusions Score; wherein the Biomarker Panel Score is obtained as per the method of claim 1; wherein the Delusions Score is calculated with a clinical rating or self-report scales; wherein the Grooming Score is calculated with a rating scale; assessing the level of delusions of the individual by comparing the individual's Up-Delusions Score to a reference Up-Delusions Score; administering a treatment for delusions to the individual when the individual's Up-Delusions Score is greater than a reference Up-Suicide Score; and monitoring the individual's response to a treatment for delusions by determining changes in the Up-Delusions Score after initiating a treatment.

A seventh embodiment is a method for assessing and/or treating schizophrenia and other psychotic disorders in general, in particular hallucinations and risk of developing hallucinations in an individual, comprising the steps of: (a) obtaining a biological sample from an individual and quantifying the amounts of one or more RNA biomarkers in the biological sample, to create at least one panel of RNA biomarkers, (b) quantifying the amounts of the RNA biomarkers in the at least one panel in a clinically relevant population to generate a reference expression level for the RNA biomarkers in a panel of RNA biomarkers; (c) comparing the amounts of the biomarkers in the biological sample from the individual with the amounts of the RNA biomarkers present in the reference standard to generate a score for each biomarker a first panel and a second panel; wherein the biomarkers in the first panel comprise one or more of the following RNA biomarkers: (PRICKLE1), (NCAM1), (B3GALT5), (ARHGAP18), (PTP4A2), Acylphosphatase 2 (ACYP2), Reticulon 4 (RTN4), Cullin 4A (CUL4A), Zinc Finger E-Box Binding Homeobox 2 (ZEB2), Dystonin (DST), and Discs Large MAGUK Scaffold Protein 1 (DLG1), wherein the expression level of the biomarker(s) in the sample is increased relative to a reference expression level, denoting hallucinations or an increased risk for developing hallucinations; and wherein the biomarkers in the second panel comprise one or more of the following RNA biomarkers: (PRL), (SERPING1), Ectonucleotide Pyrophosphatase/Phosphodiesterase 2 (ENPP2), (LAMA4), (KCNV1), Catenin Delta 1 (CTNND1), and FAT Atypical Cadherin 4 (FAT4), wherein the expression level of the RNA biomarker(s) in the sample is decreased relative to a reference expression level of the RNA biomarkers in the panel, denoting hallucinations or an increased risk for developing hallucinations; (d) generating a score for the panel of RNA biomarkers, based on the scores of the biomarker(s) in the panel; (e) determining a reference score for the panel in a clinically normal relevant population; (f) identifying a difference between the score of the panel of biomarker(s) in the sample and the reference score of the panel of biomarker(s); (g) identifying the individual as manifesting hallucinations or of having an elevated risk for developing hallucinations, based on the difference between the biomarker panel score of the individual relative to the biomarker panel score of the reference; (h) treating the individual identified as having hallucinations or an elevated risk of hallucinations with one or more of the following: 1) a treatment based on clinical practice guidelines, 2) administering a therapeutically effective amount of a therapeutic drug (s), selected based on the specific biomarkers whose scores indicate that they are changed in the individual compared to a reference standard.

An eighth embodiment is any embodiment from the first through the seventh embodiments wherein the biomarkers are quantified in samples taken on two or more occasions from the individual. As noted earlier, samples can be taken from tissue or any bodily fluid harboring RNA biomarkers.

A ninth embodiment is a method of the seventh embodiment wherein each biomarker is assigned a weighted coefficient based on the biomarkers importance in in assessing and predicting hallucinations risk; and the biomarker panel score is based on the weighted coefficients of each of the biomarkers.

A tenth embodiment is any embodiment from the seventh through the ninth embodiments, wherein the one or more therapeutic is one or more compounds selected from the group consisting of: clioquinol, pirinixic acid, moxisylyte, Prestwick-685, exemestane, azacytidine, C-75, estradiol, tetraethylenepentamine, sparteine, guanethidine, idoxuridine, gliclazide, nitrendipine, N-acetyl-L-aspartic acid, sulfanilamide, doxazosin, pimozide, Proscillaridin, oxetacaine, BRD-K71489689, trichostatin A, A443654, AG 825, Proscillaridin A, Ala-Ala-Phe-CMK, Fluocinolone acetonide, manumycin A, curcumin, BRD-K68548958, CHR 2797, Tyrphostin AG 1478, Wortmannin, HY-50878, 598226, 51003, BRD-A52530684, CGP-60474, Buparlisib, and AS-601245.

An eleventh embodiment is the method according to the seventh embodiment wherein the individual is male, and the biomarkers in a first panel (a) comprise one or more biomarkers selected from the group consisting of: (SH3PXD2A), Zinc Finger E-Box Binding Homeobox 2 (ZEB2), (PRICKLE1), (ARHGAP18), Acylphosphatase 2 (ACYP2), Reticulon 4 (RTN4), and Dystonin (DST), wherein the expression level of the biomarker(s) in the sample is increased relative to a reference expression level, denoting increased hallucinations; and biomarkers in a second panel (b) comprise one or more biomarkers selected from the group consisting of: (PRL), (SERPING1), Ectonucleotide Pyrophosphatase/Phosphodiesterase 2 (ENPP2), (KCNV1), Mab-21 Like 1 (MAB21L1), Catenin Delta 1 (CTNND1), and FAT Atypical Cadherin 4 (FAT4), wherein the expression level of the biomarker(s) in the sample is decreased relative to a reference expression level, denoting increased hallucinations. When the individual is an male the at least one therapeutic compound selected from the group consisting of: digoxigenin, doxazosin, meptazinol, promethazine, cefixime, velnacrine, cetirizine, eldeline, atropine oxide, clioquinol, nicotinic acid, clioquinol, galantamine, rolitetracycline, betahistine, sulconazole, monocrotaline, lanatoside C, Prestwick-1084, Naftidrofuryl, sulfachlorpyridazine, helveticoside, bezafibrate, mifepristone, trichostatin A, manumycin A, NCGC00189555-02, Buparlisib, linifanib, AZD-7762, Dinaciclib, Piretanide, KN-62, Fluticasone propionate, JAK3 Inhibitor VI, Sarmentogenin, Digoxin, Megestrol acetate, Oxymetazoline hydrochloride, U-0126, Tracazolate hydrochloride, Flufenamic acid, Fenofibrate, and U 99194 maleate, may be particularly effective in treating the individual, although any therapeutically effective drug may be used to treat the individual.

A twelfth embodiment is the method according to the sixth embodiment wherein the individual is female, and the biomarkers in a first panel (a) comprise one or more biomarkers selected from the group consisting of: (CELSR2), (KALRN), (B3GALT5), Protein Phosphatase 3 Catalytic Subunit Beta (PPP3CB), (ZFR), (THNSL1), (TNIK), Nuclear Receptor Subfamily 4 Group A Member 2 (NR4A2), Zinc Finger E-Box Binding Homeobox 2 (ZEB2), and (TNIK), wherein the expression level of the biomarker(s) in the sample is increased relative to a reference expression level, denoting increased hallucinations; and biomarkers in a second panel (b) comprise one or more biomarkers selected from the group consisting of GNAS Complex Locus (GNAS), and Catenin Delta 1 (CTNND1), wherein the expression level of the biomarker(s) in the sample is decreased relative to a reference expression level, denoting increased hallucinations. When the individual is a female at least one therapeutic compound selected from the group consisting of: proglumide, quinethazone, esculin, MG-262, GW-8510, haloperidol, guanethidine, deferoxamine, citiolone, meteneprost, amylocaine, CP-944629, Clemizole, IC-86621, Nortriptyline, CP-944629, Tanespimycin, Prestwick-674, 0317956-0000, and Pioglitazone, may be particularly effective in treating the individual, although any therapeutically effective drug may be used to treat the individual.

Still another embodiment is a method of treating and or accessing schizophrenia and other psychotic disorders in general, and hallucinations in particular, in an individual, comprising: calculating combined biomarkers and clinical information Up-based on the equation: (Biomarker Panel Score)+(Hallucinations Score)−(Grooming Score)=Up-Hallucinations Score; wherein the Biomarker Panel Score is obtained as per the seventh embodiment; wherein the Hallucinations Score is calculated with a clinical rating or self-report scales; wherein the Grooming Score is calculated with a rating scale; assessing the level of hallucinations of the individual by comparing the individual's Up-Hallucinations Score to a reference Up-Hallucinations Score; administering a treatment for hallucinations to the individual when the individual's Up-Hallucinations Score is greater than a reference Up-Suicide Score; and monitoring the individual's response to a treatment for hallucinations by determining changes in the Up-Hallucinations Score after initiating a treatment.

### BRIEF DESCRIPTION OF THE FIGURES

FIG. 1A. Cohorts used in study, depicting flow of discovery, prioritization, and validation of biomarkers for delusions.

FIG. 1 B. Discovery cohort longitudinal within-subject analysis. Phchp ###is study ID for each subject. V #denotes visit number.

FIG. 1 C. The clinical phenotypic measure (item from PANSS) used for delusions.

FIG. 1 D. Prioritization using Convergent Functional Genomics.

FIG. 1 E. Validation in independent cohort of psychiatric patients with clinically severe psychosis.

FIG. 1F. Schematic illustrating the results from the first 3 steps for delusions biomarkers.

FIG. 1 G. Cohorts used in study, depicting flow of discovery, prioritization, and validation of biomarkers for hallucinations.

FIG. 1 H. Discovery cohort longitudinal within-subject analysis. Phchp ###is the study identifier (ID) for each subject. V #denotes visit number.

FIG. 1 I. The clinical phenotypic measure (item from PANSS) used for discovery.

FIG. 1 J. Prioritization using Convergent Functional Genomics.

FIG. 1 K. Validation in independent cohort of psychiatric patients with clinically severe psychosis.

FIG. 1 L. Schematic illustrating the results from the first 3 steps for hallucinations biomarkers.

FIG. 2A. Top Predictive Biomarkers for high delusions state predictions (p1≥4).

FIG. 2B. Top Predictive Biomarkers for delusions trait predictions first year.

FIG. 2C. Top Predictive Biomarkers for delusion trait predictions all future years.

FIG. 2D. Top Predictive Biomarkers for high hallucinations state predictions.

FIG. 2E. Top Predictive Biomarkers for hallucinations trait predictions first year.

FIG. 2F. Top Predictive Biomarkers for hallucinations trait predictions all future years.

FIG. 3A. Network analysis of top candidate biomarkers, delusions.

FIG. 3A. Network analysis of top candidate biomarkers, hallucinations.

FIG. 4A. Example of a possible report to communicate to clinicians—delusion severity.

FIG. 4B. Example of a possible report to communicate to clinicians—hallucinations severity.

### DETAILED DESCRIPTION

We endeavored to find objective blood biomarkers for hallucinations and delusions, two core positive psychotic symptoms that are transdiagnostic, and sought to see whether these biomarkers can be used to track and predict clinical course. Our initial work over a decade ago (Kurian, Le-Niculescu et al. 2009)′ indicated that may be possible. We now used a more advanced methodology, with a longitudinal within-subject design for discovery, followed by prioritization, validation, and testing in independent cohorts. This comprehensive four-step approach is similar to the one used in our more recent studies on mood disorders, memory, stress, pain, and delusions. Provided here are newly identified blood gene expression biomarkers for hallucinations, and for delusions. These biomarkers opened a window into disease biology and core gene networks involved. Second, they permit objective assessment of state severity, short-term risk, and long-term risk. Third, they were used for drug repurposing. Lastly, we provide an example of how a precision medicine report for a patient would look, with objective scores for severity, and list of prioritized suggested medications. Such tools can be used for informing assessment, treatment choices, and monitoring response to treatment, and ultimately in prevention. Their integration in routine clinical practice and new drug development, including use and development of psychedelic drugs, can be transformative in an area that is in great need of progress.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure is generally directed at methods for assessing psychosis and/or schizophrenia and early identification of risk for future schizophrenia, as well as methods for matching patients and drugs for prevention and mitigation of schizophrenia and symptoms such as hallucinations and/or delusion, and for monitoring response to treatment. The methods may further include the generation of a report providing a risk score and/or personalized treatment options. Further, the present disclosure generally is directed to drugs for mitigating schizophrenia in subjects. Particular drugs have been found that can mitigate schizophrenia in subjects universally; that is, drugs that can be used for mitigating schizophrenia across psychiatric diagnoses and genders. Some drugs, however, have been found that can be used more effectively for mitigating schizophrenia dependent on gender, psychiatric diagnoses, and combinations thereof.

In additional embodiments, the present disclosure is directed to blood gene expression biomarkers that are more universal in nature; that is, blood biomarkers that can be used for predicting schizophrenia across psychiatric diagnoses and genders. Accordingly, a longitudinal within-participant design and large cohorts were used.

Additionally, subtypes of schizophrenia were identified based on mental state (hallucinations, delusions, psychosis) at the time that schizophrenia was manifest.

### Materials and Methods

### Cohorts

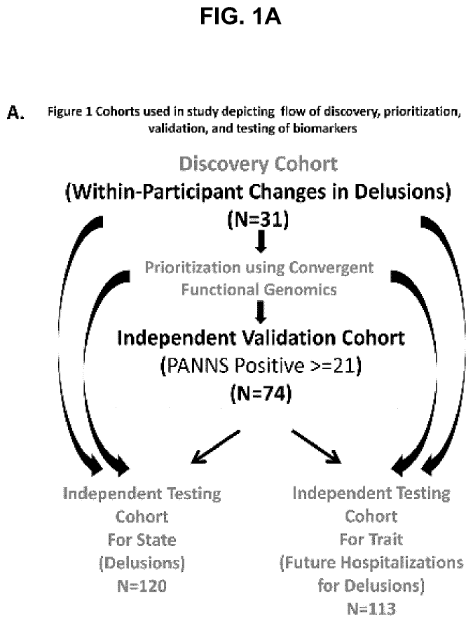

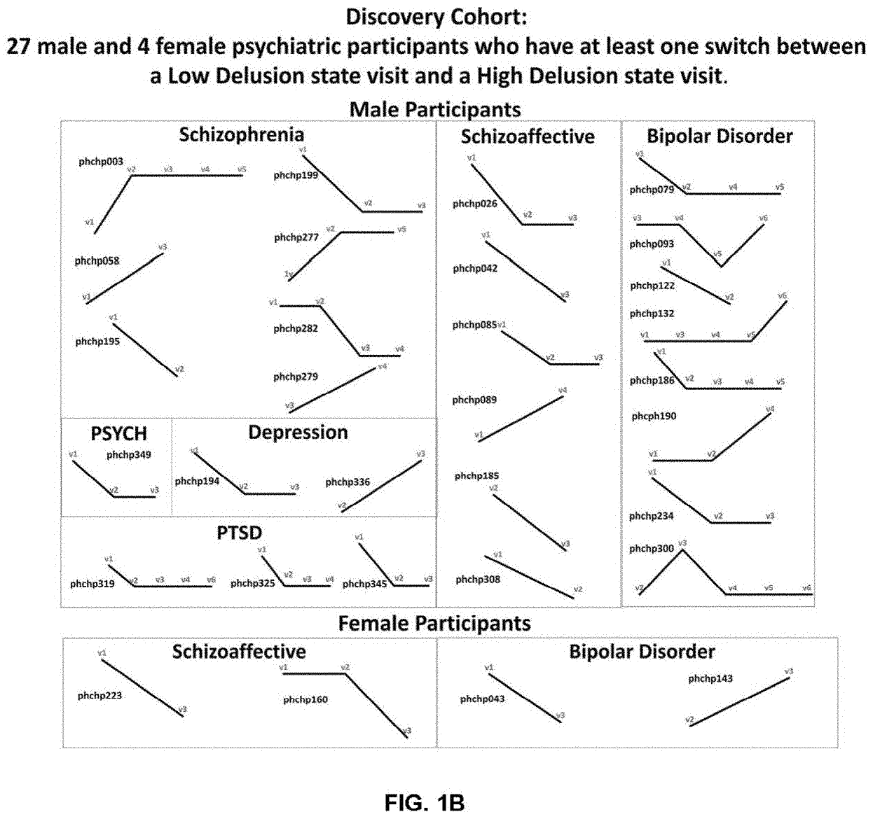

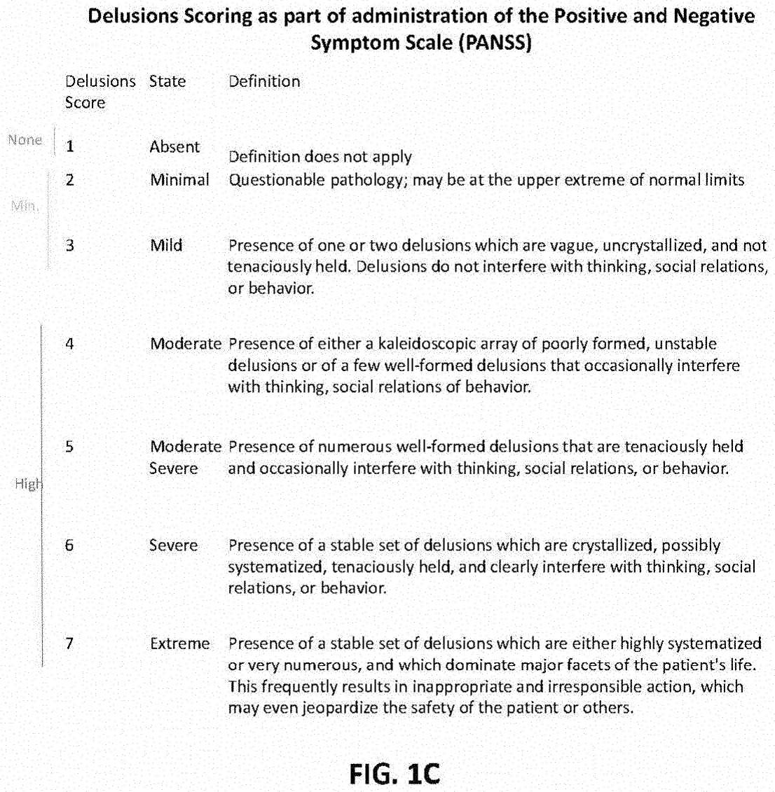

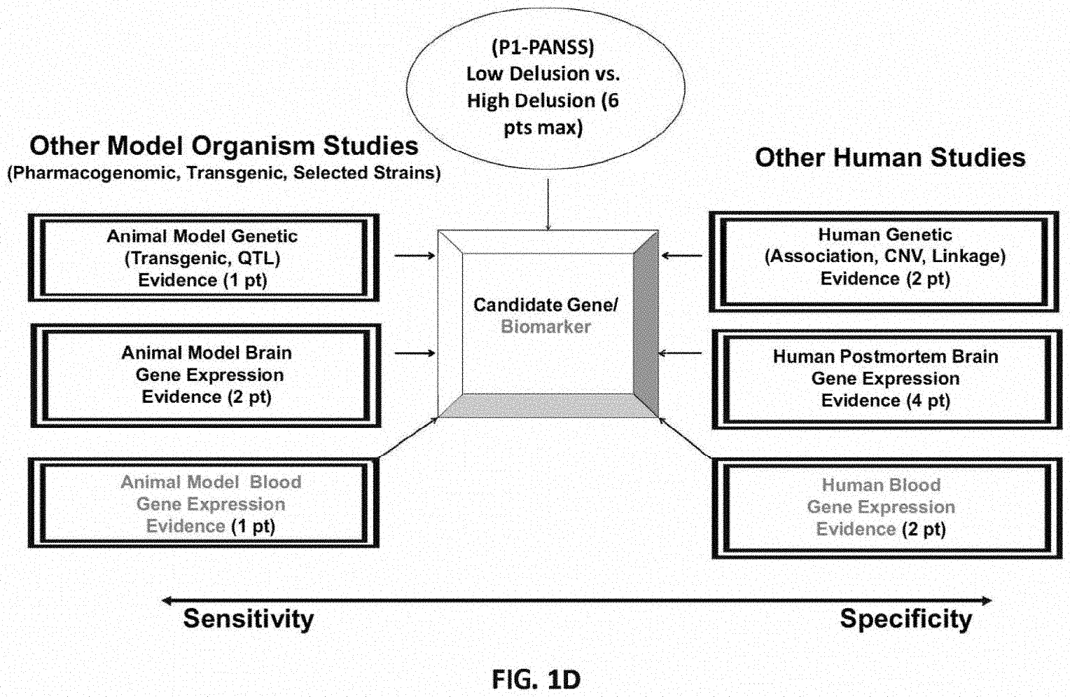

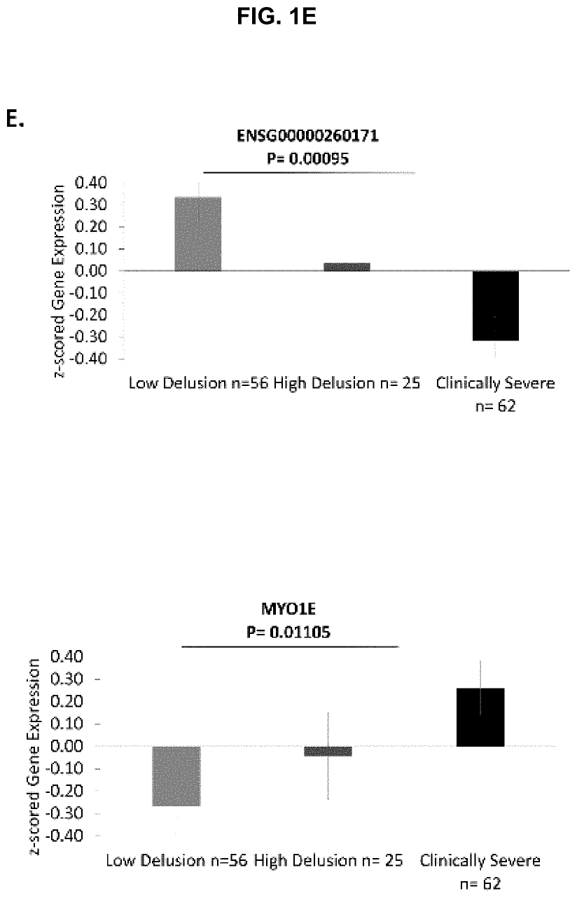

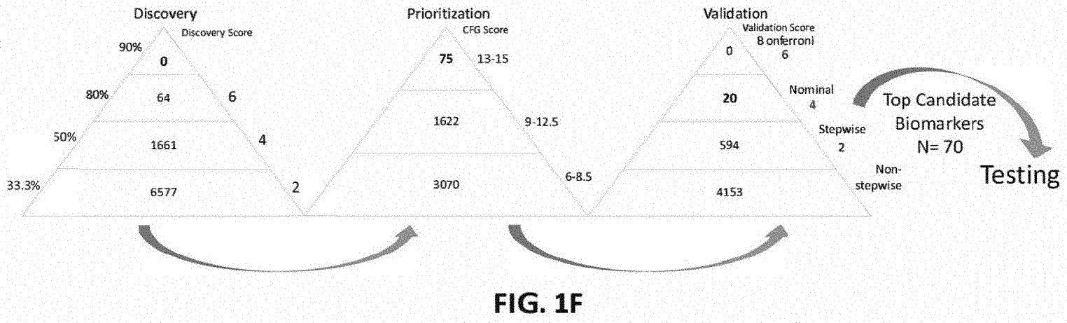

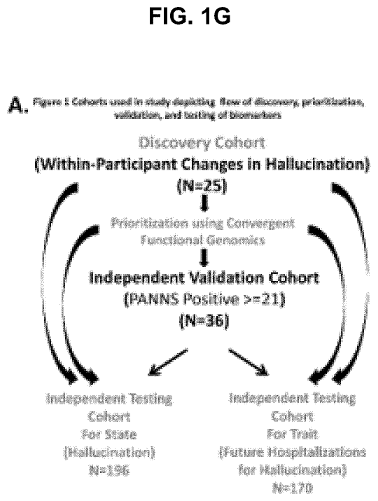

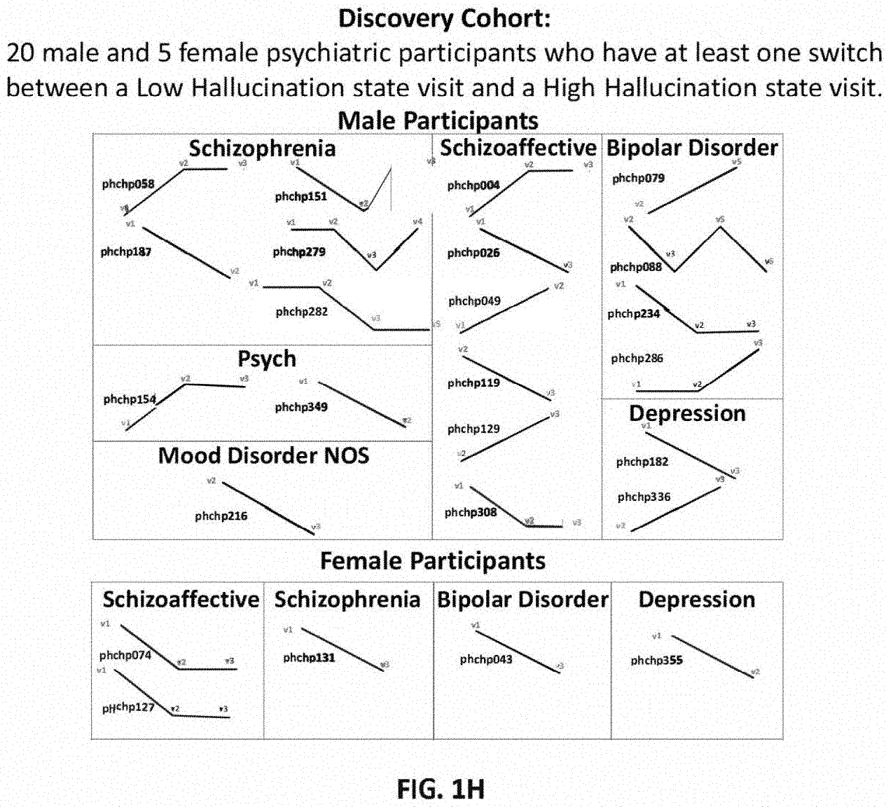

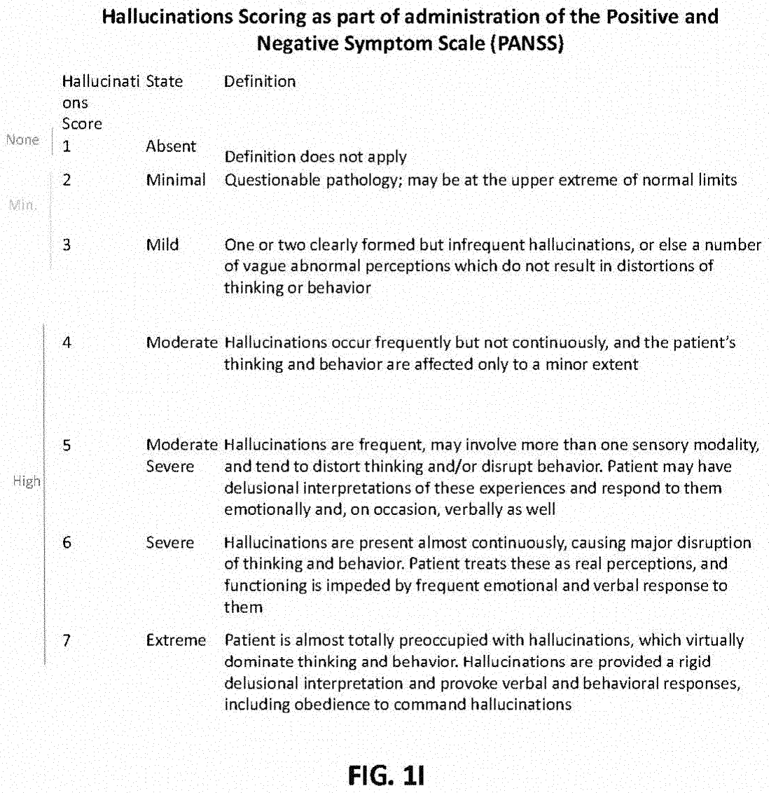

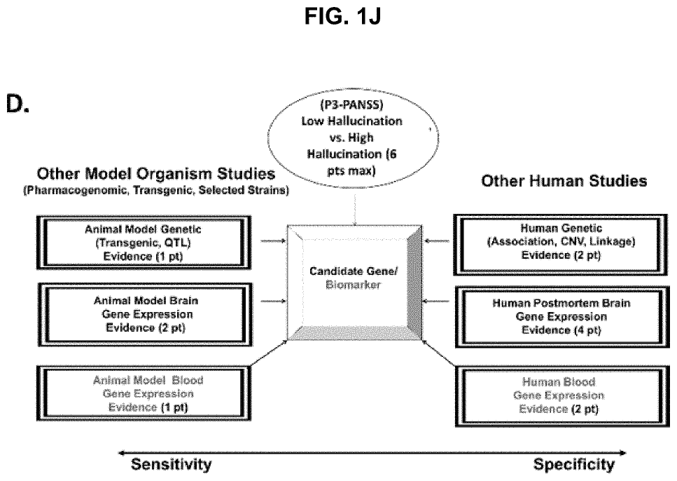

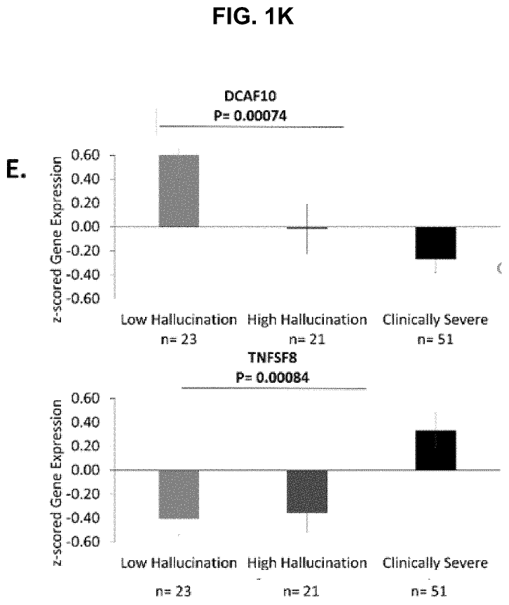

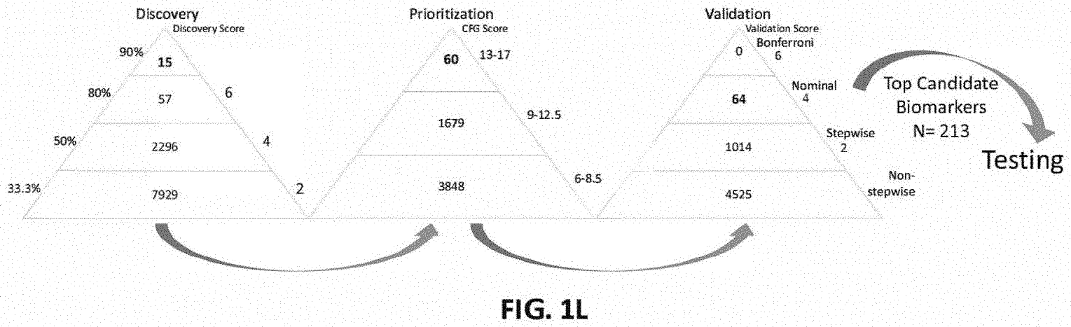

For each of the two core schizophrenia psychotic symptoms, delusions and hallucinations, we used three independent cohorts:
1. discovery (a longitudinal psychiatric subjects cohort with diametric changes in state from at least two consecutive testing visits);
2. validation (an independent psychiatric subjects cohort with clinically severe psychosis); and
3. testing (an independent psychiatric subjects test cohort for predicting psychosis state, and for predicting future hospitalizations with psychotic symptoms) (FIG. 1 and Table 1).

Similar to our previous studies, the live psychiatric subjects are part of a larger longitudinal cohort of adults that we are continuously collecting. Subjects are recruited primarily from the patient population at the Indianapolis VA Medical Center. All subjects understood and signed informed consent forms detailing the research goals, procedure, caveats and safeguards, per IRB approved protocol. Subjects completed diagnostic assessments by structured clinical interviews. They had an initial testing visit in the lab or on the inpatient psychiatric unit, followed by up to six testing visits, 3-6 months apart or whenever a new psychiatric hospitalization occurred. At each testing visit, they received a series of psychiatric rating scales, and their blood was drawn. The rating scales included the clinically used Positive and Negative Symptoms Scale (PANSS), containing the PANSS Positive sub-scale, that measures positive psychotic symptoms including delusions and hallucinations, as well as a new visual analog scale for assessing psychosis state, which provides a score that is the average of several items. This scale provides a score for psychosis state at a particular moment in time.

At each visit, we collected whole blood (5 ml) in two RNA-stabilizing PAXgene tubes, labeled with an anonymized study ID number, and stored at −80° C. in a locked freezer until the time of future processing. Whole-blood RNA was extracted for microarray gene expression studies from the PAXgene tubes, as detailed below.

For this study, for the delusions biomarker part, our within-subject discovery cohort, from which the biomarker data were derived, consisted of 31 subjects with psychiatric disorders and multiple testing visits, who each had at least one diametric change in PANSS item P1 Delusion score from no symptoms (score=1) to high symptoms (score>=4), or vice versa, from one testing visit to another. There a total of 95 blood samples for subsequent gene expression microarray studies (FIG. 1, Table 1).

For the hallucinations biomarker part, our within-subject discovery cohort, from which the biomarker data were derived, consisted of 25 subjects with psychiatric disorders and multiple testing visits, who each had at least one diametric change in PANSS item P3 Hallucinations score from no symptoms (score=1) to high symptoms (score>=4), or vice versa, from one testing visit to another. There a total of 65 blood samples for subsequent gene expression microarray studies (FIG. 1, Table 1).

Our independent validation cohort, in which the top biomarker findings were validated for being even more changed in expression, consisted of 43 subjects for delusions, and 36 subjects for hallucinations, with clinically severe psychosis (Table 1).

Our independent test cohort for predicting consisted of 120 subjects for delusions, and 196 subjects for hallucinations (FIG. 1 and Table 1).

Medications. The subjects in the discovery cohort were all diagnosed with various psychiatric disorders (Table 1) and had various medical co-morbidities. Their medications were listed in their electronic medical records, and documented by us at the time of each testing visit. Medications can have a strong influence on gene expression. However, there was no consistent pattern of any particular type of medication, as our subjects were on a wide variety of different medications, psychiatric and non-psychiatric. Furthermore, the independent validation and testing cohort's gene expression data was Z-scored by gender and by diagnosis before being combined, to normalize for any such effects. Some subjects may be non-compliant with their treatment and may thus have changes in medications or drug of abuse not reflected in their medical records. That being said, our goal is to find biomarkers that track psychosis, regardless if the reason for it is endogenous biology or it is driven by medications or drugs. Moreover, the prioritization step that occurs after discovery is based on a field-wide convergence with literature that includes genetic data and animal model data, that are unrelated to medication effects. Overall, the discovery, validation and replication by testing in independent cohorts of the biomarkers, with our design, occurs despite the subjects having different genders, diagnoses, being on various different medications, and other lifestyle variables.

### Blood Gene Expression Experiments

RNA extraction. Whole blood (2.5 ml) was collected into each PaxGene tube by routine venipuncture. PaxGene tubes contain proprietary reagents for the stabilization of RNA. RNA was extracted and processed as previously described^{2,3,4}.

Microarrays. Microarray work was carried out using previously described methodology^{2,3,4,5}.

Of note, all genomic data was normalized (RMA for technical variability, then z-scoring for biological variability), by gender and psychiatric diagnosis, before being combined and analyzed.

### Biomarkers

### Step 1: Discovery

We have used the subject's score from a visual-analog scale PANSS scale P1 and P3 items, assessed at the time of blood collection (FIG. 1). We analyzed gene expression differences between visits with low psychosis (defined as a score of 1) and visits with high psychosis (defined as a score of >=4), using a powerful within-subject design, then an across-subjects summation (FIG. 1).

We analyzed the data in two ways: an Absent-Present (AP) approach, and a differential expression (DE) approach, as in previous work by us on suicide biomarkers²⁻⁴. The AP approach may capture turning on and off of genes, and the DE approach may capture gradual changes in expression. Analyses were performed as previously described³⁻⁵. In brief, we imported all Affymetrix microarray data as CEL. files into Partek Genomic Suites 6.6 software package (Partek Incorporated, St Louis, Mich., USA). Using only the perfect match values, we ran a robust multi-array analysis (RMA) by gender and diagnosis, background corrected with quantile normalization and a median polish probeset summarization of all chips, to obtain the normalized expression levels of all probesets for each chip. Then, to establish a list of differentially expressed probesets we conducted a within-subject analysis, using a fold change in expression of at least 1.2 between consecutive high- and low psychosis visits within each subject. Probesets that have a 1.2-fold change are then assigned either a 1 (increased in high psychosis) or a −1 (decreased in high psychosis) in each comparison. Fold changes between 1.1 and 1.2 are given 0.5, and fold changes less than 1.1 are given 0. These values were then summed for each probeset across all the comparisons and subjects, yielding a range of raw scores. The probesets above the 33.3% of raw scores were carried forward in analyses (FIG. 1), and received an internal score of 2 points; those above 50% 4 points, and those above 80% 6 points^{3 4 5}. We have developed in our labs R scripts to automate and conduct all these large dataset analyses in bulk, checked against human manual scoring⁵.

Gene Symbol for the probesets were identified using NetAffyx (Affymetrix) for Affymetrix HG-U133 Plus 2.0 GeneChips, followed by GeneCards to confirm the primary gene symbol. In addition, for those probesets that were not assigned a gene symbol by NetAffyx, we used GeneAnnot (https://genecards.weizmann.ac.il/geneannot/index.shtml), or if need be UCSC (https://genome.ucsc.edu), to obtain gene symbol for these uncharacterized probesets, followed by GeneCard. Genes were then scored using our manually curated CFG databases as described below (FIG. 1D).

### Step 2: Prioritization Using Convergent Functional Genomics (CFG)

Databases. We have established in our laboratory (Laboratory of Neurophenomics, www.neurophenomics.info) manually curated databases of the human gene expression/protein expression studies (postmortem brain, peripheral tissue/fluids: CSF, blood and cell cultures), human genetic studies (association, copy number variations and linkage), and animal model gene expression and genetic studies, published to date on psychiatric disorders. Only findings deemed statistically scientifically using particular experimental design and thresholds are included in the databases. Our databases include only primary literature data and do not include review papers or other secondary data integration analyses to avoid redundancy and circularity. We also favored unbiased discovery studies over candidate genes hypothesis-driven studies. These large and constantly updated databases have been used in our CFG (Convergent Functional Genomics) cross validation and prioritization platform (FIG. 1).

### Step 3: Validation Analyses

We examined which of the top candidate genes (score of 6 or above after the first two steps), were stepwise changed in expression from the clinically depressed validation group to the low psychosis discovery group to the high psychosis discovery group to the clinically psychotic validation group. A total score of 6 or above after the first two steps permits the inclusion of potentially novel genes with maximal internal score of 6 from Discovery but no external evidence CFG score from Prioritization.

The AP derived and DE derived lists of genes were combined, and the gene expression data corresponding to them was used for the validation analysis. We transferred the log transformed expression data to an Excel sheet, and non-log transformed the data by taking 2 to the power of the transformed expression value. We then Z-scored the values by gender and diagnosis. We then imported the Excel sheets with the Z-scored by gender and diagnosis expression data into Partek, and statistical analyses were performed using a one-way ANOVA for the stepwise changed probesets, and also did a stringent Bonferroni correction for all the probesets tested in ANOVA (FIG. 1).

Top Candidate Biomarkers (after the First 3 Steps)

Adding the scores from the first three steps into an overal convergent functional evidence (CFE) score (FIG. 1), we ended up with a list of 70 top candidate biomarkers for delusions, and 213 for hallucinations (Table 2 A and 2B). These top candidate biomarkers were carried forward into additional testing for clinical utility.

### Networks

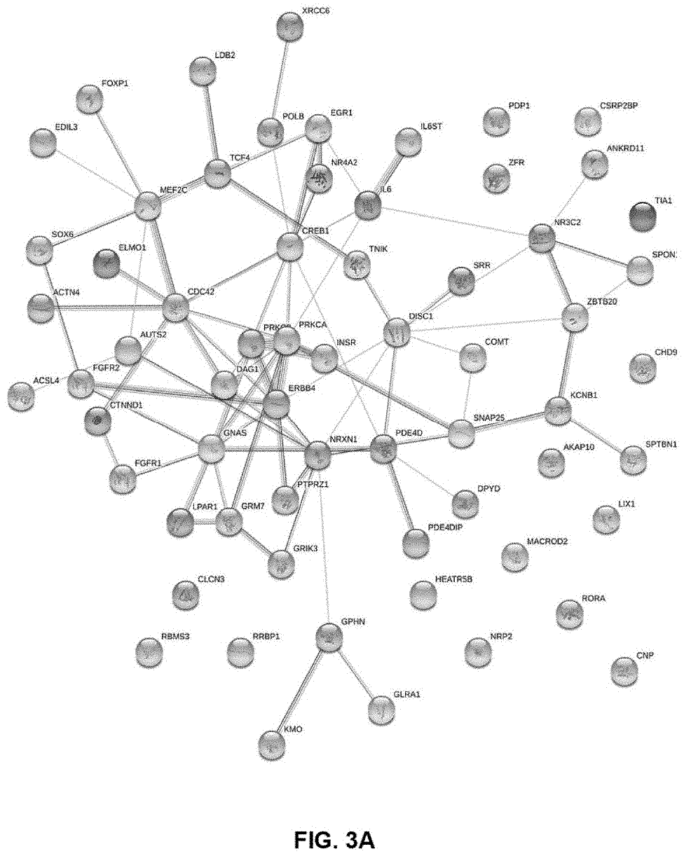

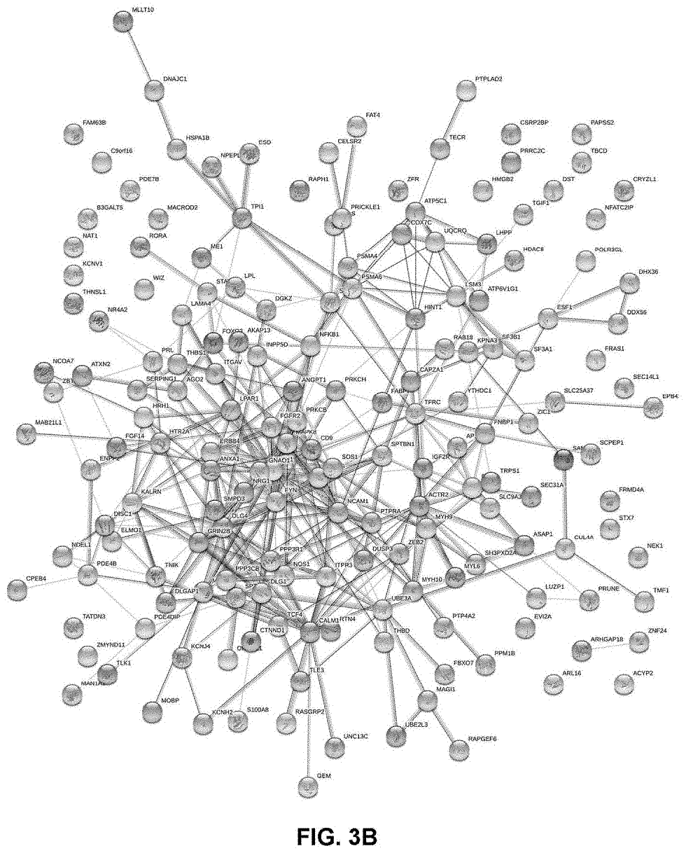

For network analyses we performed STRING Interaction network (https://string-db.org) by inputting the genes into the search window and performed Multiple Proteins Homo sapiens analysis. (FIG. 3).

### Testing for Clinical Utility in Independent Cohorts

We tested in independent cohorts of psychiatric patients the ability of each of the top candidate biomarkers to assess state severity, and predict trait risk (future hospitalizations with psychosis). We conducted our analyses across all patients, as well as personalized by gender and diagnosis.

The test cohort for predicting psychosis (delusions, hallucinations) were assembled out of data that was RMA normalized by gender and diagnosis. The cohort was completely independent from the discovery and validation cohorts, there was no subject overlap with them. Individual markers used for predictions were Z scored by gender and diagnosis, to be able to combine different biomarkers into panels and to avoid potential artefacts due to different ranges of expression in different gender and diagnoses. For panels, biomarkers were combined by simple summation of the increased risk biomarkers minus the decreased risk biomarkers. Predictions were performed using R-studio (opensource). For cross-sectional analyses, we used biomarker expression levels, z-scored by gender and diagnosis. For longitudinal analyses, we combined four measures: biomarker expression levels, slope (defined as ratio of levels at current testing visit vs. previous visit, divided by time between visits), maximum levels (at any of the current or past visits), and maximum slope (between any adjacent current or past visits). For decreased biomarkers, we used the minimum rather than the maximum for level calculations. All four measures were Z-scored, then combined in an additive fashion into a single measure. The longitudinal analysis was carried out in a sub-cohort of the testing cohort consisting of subjects that had at least two subject visits (timepoints).

Predicting State Severity. Receiver-operating characteristic (ROC) analyses between marker levels and psychosis state were performed.

Predicting Trait—Future Psychiatric Hospitalization with Psychosis as a Symptom/Reason for Admission. We conducted analyses for predicting future psychiatric hospitalizations with psychosis as a symptom/reason for admission in the first year following each testing visit, in subjects that had at least one year of follow-up in the VA system, in which we have access to complete electronic medical records. ROC analyses between biomarkers measures (cross-sectional, longitudinal) at a specific testing visit and future hospitalizations within the first year were performed. A Cox regression was performed for all future hospitalizations. The odds ratio was calculated such that a value greater than 1 always indicates increased risk for hospitalization, regardless if the biomarker is increased or decreased in expression.

### Therapeutics

Pharmacogenomics. We analyzed which of the top biomarkers for psychosis after Steps 1-4 are known to be changed in expression by existing drugs in a direction opposite to the one in disease, using our CFG databases, and using Ingenuity Drugs analyses (Table 3).

New drug discovery/repurposing. We also analyzed which drugs and natural compounds are an opposite match for the gene expression signatures of our top biomarkers, using the Connectivity Map (https://portals.broadinstitute.org, Broad Institute, MIT) (Table 4). Of note, not all the probesets from the HG-U133 Plus 2.0 array we used were present in the HGU-133A array used for the Connectivity Map. We stayed with exact probeset level matches, not gene level imputation. We also used the NIH LINCS database to conduct similar analyses, at a gene level.

### Report Generation

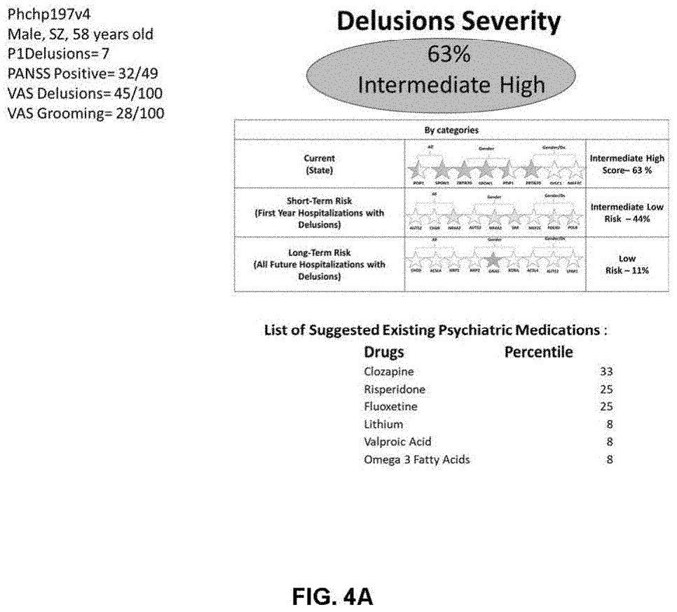

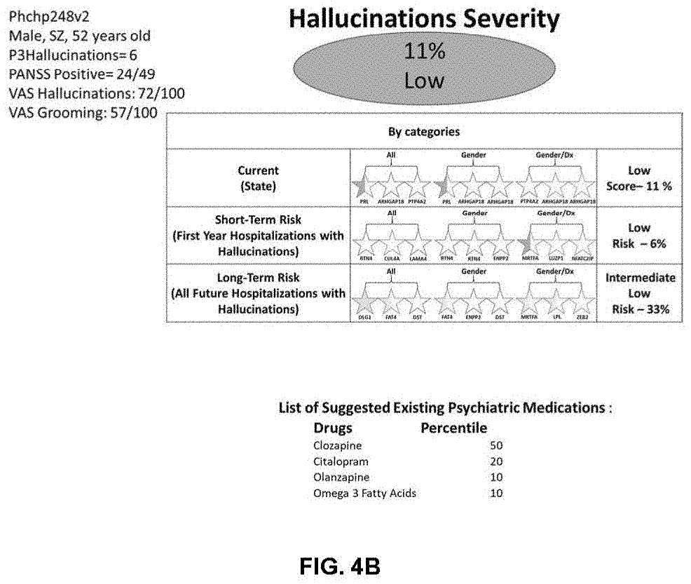

We present an example of how a report to doctors might look, using the described methods and identified biomarkers. Out of a dataset of 794 subject visits, we chose, as case studies, a visit from a patient with self-reported high delusions, and one from a patient with self-reported high hallucinations (FIG. 4).

For each biomarker in the panel, we also have a list of existing psychiatric medications that modulate the expression of the biomarker in the direction of high psychosis. Each medication got a score of 1 or 0 whether it modulated a particular biomarker in the panel or not, and that score is multiplied with the risk score of the biomarker, i.e. 1 or 0.5 or 0. A medication can modulate more than one biomarker. We then calculated an average score for each medication based on its effects on all the biomarkers in the panel, and multiplied that by 100, resulting in a % score for each medication. Thus, psychiatric medications are matched to the patient and ranked in order of impact on the panel.

TABLE 1
Demographics of patient cohorts used.
A. Delusions, B. Hallucinations

| | | | | | |
|---|---|---|---|---|---|
| | | | | Age in years at | |
| Number of | | | | time of lab visit | |
| subjects | | | | Mean | |
| (number | | | | (SD) | T-test for age at |
| of visits) | Gender | Diagnosis | Ethnicity | (Range) | time of lab visit |

A. Delusions
Discovery

| | | | | | |
|---|---|---|---|---|---|
| Discovery Cohort | 31 (with | Male | BP = 10 (35) | AA = 10 (28) | 49.45 |
| (Longitudinal Within- | 95 visits) | 27 (86) | MDD = 2 (5) | EA = 21 (67) | (7.38) |
| Subject Changes in | | Female | PSYCH = 1 (3) | | (30-63) |
| Delusion) | | 4 (9) | PTSD = 3 (12) | | |
| P1 >= 4 | | | SZ = 7 (21) | | |
| high Delusions | | | SZA = 8 (19) | | |
| P1 <= 1 | | | | | |
| No Delusions | | | | | |

Validation

| Independent Validation | 43 (with | Male | BP = 7(8) | AA = 16(25) | 46.11 | T-test for age |
|---|---|---|---|---|---|---|
| Cohort for | 62 visits) | 36 (52) | MOOD = 1(2) | EA = 27(37) | (11.55) | Validation (Clinical) vs |
| Gene Expression | | Female | PSYCH = 1 (I) | | (22-62) | Stepwise Discovery |
| (P1 >= 4 | | 7 (10) | PTSD = 3(6) | | | (High and Low) = .11 |
| high Delusion | | | SZ = 15(23) | | | |
| and PANSS Positive | | | SZA = 16(22) | | | |
| Total >= 21) | | | | | | |
| Stepwise Discovery | Stepwise Discovery High | | | | | |
| Cohorts Used for | 23 (with | Male | BP = 6(6) | AA = 7(8) | 48.52 | |
| Validation | 25 visits) | 21 (23) | MDD = 1(I) | EA = 16(17) | (6.87) | |
| P1 >= 4 | | Female | PTSD = 3 (3) | | (33-61) | |
| high Delusions | | 2 (2) | SZ = 6(8) | | | |
| & PANSS Positive <21 | | | SZA = 7(7) | | | |
| P1 <= 1 | Stepwise Discovery Low | | | | | |
| No Delusions | 31 (with | Male | BP = 10(23) | AA = 10(15) | 49 | |
| | 56 visits) | 27 (52) | MDD = 2(3) | EA = 21(41) | (7.1) | |
| | | Female | PSYCH = 1 (2) | | (31-63) | |
| | | 4(4) | PTSD = 3 (9) | | | |
| | | | SZ = 7(9) | | | |
| | | | SZA = 8(10) | | | |

Testing

| | | | | | | |
|---|---|---|---|---|---|---|
| Independent Testing | 120 (with | Male | BP = 51 (147) | AA = 25 (63) | All 51 | T-test for age |
| Cohort For | 315 visits) | 109 (285) | MDD = 10 (28) | EA = 94 (249) | (9.1) | High Delusions |
| Predicting State | | Female | SZ = 27 (64) | Hispanic = 1 (3) | (23-74) | (N = 36) |
| (High Delusions | | 11 (30) | SZA = 32 (76) | | High Delusions | vs. Others |
| P1 ≥ 4, P1 <= 3 | | | | | (N = 36) 51.3 | (N = 279) |
| (others) at Time of | | | | | Others | 0.8 |
| Assessment) | | | | | (N = 279) 51 | |
| Independent Testing | 113(with | Male | BP = 44 (122) | AA = 35 (87) | All = 49.6 | T-test for age |
| Cohort For | 292 visits) | 99 (253) | MDD = 9 (25) | EA = 77 (202) | (8.21) | No Hosp with |
| Predicting Trait | | Female | PSYCH = 3(7) | Hispanic = 1 (3) | (23-63) | Delusions |
| (All Future | | 14 (39) | PTSD = 1(3) | | No Hosp for Delusions | (N = 212) |
| Hospitalizations | | | SZ = 25 (60) | | (N = 212) 49.8 | vs. Hosp with |
| with Delusions | | | SZA = 31(75) | | Hosp for Delusions | Delusions |
| following | | | | | (N = 80) 49.1 | (N = 80) |
| Assessment) | | | | | | 0.5177799 |
| Independent Testing | 95 (with | Male | BP = 45 (135) | AA = 18 (48) | All = 50.9 | T-test for age |
| Cohort For | 254 visits) | 91 (243) | PSYCH = 3(7) | EA = 77 (206) | (9.21) | No Hosp with |
| Predicting Trait | | Female | SZ = 27(64) | | (23-74) | Delusions |
| (Hospitalizations | | 4 (11) | SZA = 20(48) | | No Hospitalizations | (N = 239) |
| with Delusions in the | | | | | visit for Delusions | vs. Hosp with |
| First Year Following | | | | | (N = 239) 50.9 | Delusions within |
| Assessment) | | | | | Hospitalizations | the first Year |
| | | | | | for Delusions | (N = 15) |
| | | | | | (N = 15) 50.5 | 0.815349 |

B. Hallucinations
Discovery

| | | | | | |
|---|---|---|---|---|---|
| Discovery Cohort | 25 (with | Male | BP = 5(14) | AA = 12(31) | 49.57 |
| (Longitudinal Within- | 65 visits) | 20(53) | MDD = 3(6) | EA = 13(34) | (10) |
| Subject Changes in | | Female | MOOD = 1 (2) | | (24-62) |
| Hallucination) | | 5 (12) | PSYCH = 2(5) | | |
| P3 >= 4 | | | SZ = 6(18) | | |
| high Hallucinations | | | SZA = 8(20) | | |
| P3 <= 1 | | | | | |
| No Hallucinations | | | | | |

Validation

| Independent Validation | 36 (with | Male | BP = 10 (13) | AA = 12(17) | 46.92 | T-test for age |
|---|---|---|---|---|---|---|
| Cohort for | 52 visits) | 27(39) | PTSD = 4 (6) | EA = 24(35) | (10) | Validation (Clinical) |
| Gene Expression | | Female | SZ = 12 (21) | | (22-63) | vs Stepwise Discovery |
| (P3 >= 4 | | 9 (13) | SZA = 10 (12) | | | (High and Low) = .542434 |
| high Hallucination | | | | | | |
| and PANSS Positive | | | | | | |
| Total >= 21) | | | | | | |
| Stepwise Discovery | Stepwise Discovery High | | | | | |
| Cohorts Used for | 16 (with | Male | BP = 4(5) | AA = 7(11) | 50.33 | |
| Validation | 21 visits) | 13(18) | MDD = 2(2) | EA = 9(10) | (8.5) | |
| P3 >= 4 | | Female | PSYCH = 1(2) | | (24-61) | |
| high Hallucination | | 3 (3) | SZ = 4(7) | | | |
| & PANSS Positive <21 | | | SZA = 5(5) | | | |
| P3 <= 1 | Stepwise Discovery Low | | | | | |
| No Hallucination | 19 (with | Male | BP = 5(8) | AA = 9(11) | 46.17 | |
| | 24 visits) | 15(19) | MDD = 2(2) | EA = 10(13) | (9) | |
| | | Female | MOOD = 1(I) | | (24-56) | |
| | | 4(5) | PSYCH = 1(I) | | | |
| | | | SZ = 3(3) | | | |
| | | | SZA = 7(9) | | | |

Testing

| | | | | | | |
|---|---|---|---|---|---|---|
| Independent Testing | 196 (with | Male | BP = 74 (207) | AA = 55 (135) | All 50.1 | T-test for age |
| Cohort For | 513 visits) | 162 (426) | MDD = 22 (55) | EA = 139 (372) | (9.2) | High P3 |
| Predicting State | | Female | MOOD = 6 (17) | Hispanic = 2 (6) | (20-74) | (N = 62) |
| (High Hallucination | | 34 (87) | PTSD = 25 (64) | | High P3 | vs. Others |
| P3 ≥ 4, P3 <= 3 | | | SZ = 30 (74) | | (N = 62) 50.7 | (N = 451) |
| (others) at Time of | | | SZA = 39 (96) | | Others | 0.54722 |
| Assessment) | | | | | (N = 451) 50.0 | |
| Independent Testing | 163 (with | Male | BP = 55 (165) | AA = 33 (84) | All = 50.3 | T-test for age |
| Cohort for | 437 visits) | 146 (389) | MDD = 30 (77) | EA = 130 (353) | (8.3) | No Hosp with |
| Predicting Trait | | Female | PTSD = 14 (39) | | (23-65) | Hallucination |
| (Hospitalizations with | | 17 (48) | SZ = 29 (72) | | No Hosp for P3 | (N = 415) |
| Hallucinations in the | | | SZA = 35 (84) | | (N = 415) 50.5 | vs. Hosp with |
| First Year Following | | | | | Hosp for P3 | Hallucination |
| Assessment) | | | | | (N = 22) 49.3 | within the first Year |
| | | | | | | (N = 22) |
| | | | | | | 0.509856 |
| Independent Testing | 170 (with | Male | BP = 54 (152) | AA = 51 (123) | All = 49.4 | T-test for age |
| Cohort for | 442 visits) | 153 (392) | MDD = 40 (101) | EA = 118 (316) | (7.9) | Future Hosp with |
| Predicting Trait | | Female | PTSD = 14 (39) | Hispanic = 1 (3) | (23-66) | no Hallucination |
| (Hospitalizations with | | 17 (50) | SZ = 27 (66) | | No Hosp for P3 | (N = 334) |
| Hallucinations in All | | | SZA = 35 (84) | | (N = 334) 49.5 | vs. Hallucination |
| Future Years Following | | | | | Hosp for P3 | with Hosp |
| Assessment) | | | | | (N = 109) 48.5 | (N = 109) |
| | | | | | | 0.50054434 |

Referring now to FIG. 1. Steps 1-3: Discovery, Prioritization and Validation of Biomarkers. See for examples FIGS. 1A-G Delusions; FIG. 1. H-M Hallucinations (A.) Cohorts used in study, depicting flow of discovery, prioritization, and validation of biomarkers from each step. (B.) Discovery cohort longitudinal within-subject analysis. Phchp ###is study ID for each subject. V #denotes visit number. (C.) The clinical phenotypic measure (item from PANSS) used for discovery. (D.) Prioritization with CFG for prior evidence of involvement in schizophrenia and psychotic disorders. In the prioritization step probesets are converted to their associated genes using Affymetrix annotation and GeneCards. Genes are prioritized and scored using CFG for schizophrenia/psychosis evidence with a maximum of 12 points. Genes scoring at least 6 points out of a maximum possible of 18 total discovery and prioritization scores points are carried to the validation step. (E.) Validation in independent cohort of psychiatric patients with clinically severe psychosis

TABLE 2
Top Candidate biomarkers after Steps 1-3. A. Delusions.
B. Hallucinations. “D” indicates a decrease in
RNA biomarker expression measured in a high psychosis
(delusions or hallucinations) population compared to a
population that does not have high psychosis; “I”
indicates an increase in RNA biomarker expression.

| Gene | Direction of Change |
|---|---|
| A. For Delusions (n = 70) | |
| DAG1 | D |
| LPAR1 | I |
| CTNND1 | D |
| POLB | D |
| ERBB4 | D |
| INSR | D |
| IL6 | D |
| KMO | I |
| NR4A2 | I |
| PDE4DIP | I |
| PTPRZ1 | I |
| RORA | I |
| SNAP25 | D |
| TNIK | D |
| TCF4 | I |
| ZFR | I |
| CNP | D |
| ACTN4 | I |
| AUTS2 | I |
| ACSL4 | I |
| AKAP10 | D |
| ANKRD11 | D |
| CREB1 | D |
| COMT | I |
| CDC42 | D |
| CLCN3 | I |
| CHD9 | I |
| DPYD | D |
| DISC1 | D |
| DISC1 | D |
| EGR1 | I |
| EDIL3 | I |
| ELMO1 | D |
| FGFR1 | D |
| FGFR2 | D |
| FOXP1 | I |
| FOXP1 | D |
| FOXP1 | I |
| GPHN | I |
| GRIK3 | D |
| GRM7 | I |
| GLRA1 | I |
| GNAS | I |
| HEATR5B | I |
| IL6ST | I |
| LDB2 | I |
| LIX1 | I |
| KAT14 | I |
| MACROD2 | I |
| MEF2C | D |
| NRXN1 | D |
| NRP2 | D |
| NRP2 | D |
| NR3C2 | I |
| PDE4D | D |
| KCNB1 | I |
| KCNB1 | I |
| PRKCA | I |
| PRKCB | I |
| PDP1 | I |
| RRBP1 | D |
| RBMS3 | I |
| SRR | I |
| SPTBN1 | D |
| SPON1 | D |
| SOX6 | D |
| TIA1 | D |
| XRCC6 | I |
| ZBTB20 | D |
| ZBTB20 | D |
| B. For Hallucinations (n = 213) | |
| DLG1 | I |
| PPP3CB | I |
| ERBB4 | D |
| RTN4 | I |
| CALM1 | I |
| GNAO1 | D |
| GNAS | D |
| MAN1A1 | I |
| NDEL1 | I |
| SF3B1 | I |
| NFATC2IP | I |
| ARHGAP18 | I |
| ESD | I |
| KCNV1 | D |
| LSM3 | I |
| NCAM1 | I |
| NEK1 | I |
| PDE4B | I |
| TLK1 | I |
| ZNF24 | I |
| MBP | I |
| PRKCH | D |
| THBS1 | I |
| ANXA1 | I |
| ASAP1 | D |
| ATP5C1 | I |
| ATP5C1 | I |
| ATP5C1 | I |
| ATP6V1G1 | I |
| B3GALT5 | I |
| B3GALT5 | I |
| CD9 | I |
| COX7C | I |
| CRYZL1 | I |
| DDX56 | I |
| DISC1 | I |
| DLG4 | D |
| DST | I |
| ELMO1 | D |
| ENPP2 | D |
| EVI2A | I |
| FAT4 | D |
| FGF14 | D |
| FGFR2 | D |
| FN1 | D |
| FN1 | D |
| FNBP1 | I |
| GLS | I |
| GLS | I |
| GNAS | D |
| GNAS | D |
| GNAS | D |
| GNAS | D |
| HDAC8 | I |
| HMGB2 | I |
| HTR2A | D |
| INPP5D | I |
| ITPR3 | D |
| KALRN | I |
| CSRP2BP | I |
| LAMA4 | D |
| LHPP | D |
| LPAR1 | I |
| MAB21L1 | D |
| MACROD2 | D |
| MAPK8 | I |
| MLLT10 | I |
| MKL1 | I |
| MYH10 | I |
| NCOA7 | I |
| NOS1 | I |
| ORMDL1 | I |
| PDE7B | I |
| PPM1B | I |
| PPP3CB | I |
| PPP3R1 | I |
| PRICKLE1 | I |
| PRKCB | D |
| PRKCB | D |
| PRKCB | I |
| PRRC2C | D |
| PSMA4 | I |
| RAPH1 | I |
| SAMHD1 | I |
| SEC31A | I |
| SERPING1 | D |
| SF3A1 | D |
| SF3B1 | I |
| SLC2A1 | D |
| SPTBN1 | D |
| TBCD | I |
| THBD | D |
| THNSL1 | I |
| UNC13C | I |
| ZBTB20 | I |
| ZEB2 | I |
| ZEB2 | I |
| ZIC1 | I |
| AKAP13 | I |
| AKAP13 | I |
| AKAP13 | I |
| DLGAP1 | I |
| DNAJC1 | I |
| HINT1 | I |
| HINT1 | I |
| HRH1 | I |
| NAT1 | I |
| POLR3GL | I |
| SEPTIN5 | D |
| UBE2L3 | I |
| ACTR2 | I |
| ACYP2 | I |
| ANGPT1 | D |
| AP1S2 | I |
| ARHGAP18 | I |
| ATXN2 | D |
| CAPZA1 | I |
| CELSR2 | I |
| CTNND1 | D |
| CUL4A | I |
| DGKZ | D |
| DHX36 | I |
| DUSP3 | D |
| EPB42 | D |
| ESF1 | I |
| FABP7 | I |
| FBXO7 | D |
| FOXO3 | D |
| FRAS1 | I |
| FRMD4A | I |
| FRMD4A | D |
| FYN | D |
| GRIN2B | I |
| HACD4 | I |
| ITGAV | I |
| KCNH2 | D |
| KPNA3 | D |
| LPL | I |
| LUZP1 | I |
| MAGI1 | I |
| ME1 | D |
| FAM63B | I |
| NEK1 | I |
| NFKB1 | D |
| NPEPL1 | D |
| NR4A2 | I |
| NR4A2 | I |
| NRG1 | D |
| PAPSS2 | I |
| PDE4DIP | D |
| PRL | D |
| PRUNE | D |
| PSMA6 | I |
| PTP4A2 | I |
| PPP2R4 | D |
| PPP2R4 | D |
| RAPGEF6 | D |
| RORA | I |
| RORA | D |
| RTN4 | I |
| SCPEP1 | I |
| SEC14L1 | D |
| SH3PXD2A | I |
| SLC25A37 | D |
| SLC25A37 | D |
| SLC9A3R1 | D |
| SMPD3 | D |
| SOS1 | I |
| SPTLC1 | I |
| STAT6 | D |
| TATDN3 | D |
| TCF4 | I |
| TECR | I |
| TGIF1 | I |
| TLE3 | D |
| TMF1 | I |
| TNIK | I |
| TRPS1 | D |
| UBE3A | D |
| UQCRQ | I |
| WIZ | D |
| YTHDC1 | I |
| ZMYND11 | I |
| ZNF24 | I |
| AGO2 | D |
| AGO2 | D |
| ARL16 | I |
| C9orf16 | I |
| CLTA | D |
| CLTA | D |
| CPEB4 | D |
| GEM | I |
| HSPA1B | I |
| IGF2R | D |
| IGHM | D |
| KCNJ4 | D |
| MBP | D |
| MOBP | I |
| MOBP | D |
| MYH9 | D |
| MYH9 | I |
| MYL6 | I |
| PRKCH | I |
| RAB18 | I |
| RASGRP2 | D |
| RASGRP2 | I |
| RASGRP2 | D |
| S100A8 | I |
| STX7 | I |
| TFRC | D |
| THBS1 | I |
| TPI1 | D |
| ZFR | I |

TABLE 3
Convergent Functional Evidence (CFE) for Top Biomarkers after Steps 1-4.
A. Delusions B. Hallucinations.

| | | | | | |
|---|---|---|---|---|---|
| | | | Step 2 | | Step 4 |
| | | | External Convergent | | Best Significant |
| | | Step 1 | Functional Genomics | | Prediction of High |
| | | Discovery in Blood | (CFG) Evidence | Step 3 | Delusional State |
| | | (Direction of Change | For Involvement | Validation in | ROC AUC/p-value |
| | | in High Delusions) | in Delusion | Blood ANOVA | 3 pts All |
| | | Method/Score/% | Disorders Score | p-value/Score | 2 pts Gender |
| Gene Symbol/Gene Name | Probeset | 6 pts | 12 pts | 6 pts | 1 pts Gender/Dx |

A. Top Biomarkers for Delusions.

| | | | | | |
|---|---|---|---|---|---|
| AUTS 2 | 242721_at | (I) | 10 | 0.496/0 | All |
| Activator Of Transcription And | | DE/4 | | Not Stepwise | C: (36/315) |
| Developmental Regulator AUTS 2 | | 52.78% | | | 0.62/1.14E−02 |
| | | | | | Gender |
| | | | | | Male |
| | | | | | C: (32/285) |
| | | | | | 0.62/1.28E−02 |
| | | | | | Gender/Dx |
| | | | | | M-PSYCHOSIS |
| | | | | | C: (29/138) |
| | | | | | 0.67/2.74E−03 |
| | | | | | M-SZA |
| | | | | | C: (16/76) |
| | | | | | 0.71/4.85E−03 |
| NR4A2 | 204621_s_at | (I) | 11 | 0.955/2 | |
| Nuclear Receptor Subfamily 4 | | DE/2 | | Stepwise | |
| Group A Member 2 | | 33.33% | | | |
| CHD 9 | 220586_at | (I) | 8 | 0.0121/0 | |
| Chromodomain Helicase DNA | | AP/6 | | Not Stepwise | |
| Binding Protein 9 | | 98.67% | | | |
| IL6ST | 204863_s_at | (I) | 6 | 0.968/2 | |
| Interleukin 6 Signal Transducer | | AP/6 | | Stepwise | |
| | | 84% | | | |
| MACROD2 | 242468_at | (I) | 8 | 0.108/0 | Gender |
| Mono-ADP Ribosylhydrolase 2 | | AP/6 | | Not Stepwise | Female |
| | | 100% | | | C: (4/30) |
| | | | | | 0.77/4.38E−02 |
| PDP1 | 218273_s_at | (I) | 8 | 0.972/2 | All |
| Pyruvate Dehyrogenase Phosphatase | | DE/4 | | Stepwise | L: (20/194) |
| Catalytic Subunit 1 | | 55.56% | | | 0.67/5.28E−03 |
| | | | | | Gender |
| | | | | | Female |
| | | | | | L: (2/18) |
| | | | | | 0.97/1.75E−02 |
| | | | | | Male |
| | | | | | L: (18/176) |
| | | | | | 0.64/2.72E−02 |
| | | | | | Gender/Dx |
| | | | | | F-MDD |
| | | | | | L: (2/18) |
| | | | | | 0.97/1.75E−02 |
| | | | | | M-PSYCHOSIS |
| | | | | | L: (17/80) |
| | | | | | 0.68/1.22E−02 |
| | | | | | M-SZ |
| | | | | | L: (8/36) |
| | | | | | 0.73/2.62E−02 |
| RORA RAR | 240951_at | (I) | 11 | 0.108/0 | Gender/Dx |
| Related Orphan Receptor A | | DE/4 | | Not Stepwise | M-SZ |
| | | 52.78% | | | C: (13/62) |
| | | | | | 0.65/4.93E−02 |
| FOXP1 | 223936_s_at | (I) | 10 | 0.5/0 | |
| Forkhead Box P1 | | AP/4 | | Not Stepwise | |
| | | 53.33% | | | |
| FOXP1 | 240666_at | (D) | 10 | 0.192/0 | All |
| Forkhead Box P1 | | DE/4 | | Not Stepwise | C: (36/315) |
| | | 51.35% | | | 0.6/2.25E−02 |
| | | | | | L: (20/194) |
| | | | | | 0.62/3.46E−02 |
| | | | | | Gender |
| | | | | | Female |
| | | | | | C: (4/30) |
| | | | | | 0.8/2.93E−02 |
| | | | | | Gender/Dx |
| | | | | | M-PSYCHOSIS |
| | | | | | C: (29/138) |
| | | | | | 0.63/1.86E−02 |
| | | | | | M-SZ |
| | | | | | C: (13/62) |
| | | | | | 0.7/1.57E−02 |
| | | | | | L: (8/36) |
| | | | | | 0.71/3.39E−02 |
| GNAS | 242975_s_at | (I) | 10 | 0.214/0 | |
| GNAS Complex Locus | | DE/4 | | Not Stepwise | |
| | | 58.33% | | | |
| XRCC6 | 215308_at | (I) | 8 | 0.523/2 | Gender/Dx |
| X-Ray Repair Cross | | AP/4 | | Stepwise | M-BP |
| Complementing 6 | | 66.67% | | | C: (3/147) |
| | | | | | 0.8/3.86E−02 |
| ZBTB20 | 239955_at | (D) | 10 | 0.622/2 | All |
| Zinc Finger And BTB Domain | | AP/2 | | Stepwise | C: (36/315) |
| Containing 20 | | 35.29% | | | 0.63/6.23E−03 |
| | | | | | L: (20/194) |
| | | | | | 0.65/1.57E−02 |
| | | | | | Gender |
| | | | | | Female |
| | | | | | C: (4/30) |
| | | | | | 0.81/2.55E−02 |
| | | | | | Male |
| | | | | | C: (32/285) |
| | | | | | 0.61/2.55E−02 |
| | | | | | L: (18/176) |
| | | | | | 0.65/1.79E−02 |
| | | | | | Gender/Dx |
| | | | | | F-MDD |
| | | | | | C: (3/28) |
| | | | | | 0.8/4.73E−02 |
| | | | | | M-PSYCHOSIS |
| | | | | | C: (29/138) |
| | | | | | 0.64/1.17E−02 |
| | | | | | L: (17/80) |
| | | | | | 0.65/3.37E−02 |
| | | | | | M-SZ |
| | | | | | C: (13/62) |
| | | | | | 0.74/3.58E−03 |
| | | | | | L: (8/36) |
| | | | | | 0.79/7.44E−03 |
| ACSL4 | 224091_at | (I) | 10 | 0.744/0 | Gender/Dx |
| Acyl-CoA Synthetase Long | | AP/4 | | Not Stepwise | M-SZA |
| Chain Family Member 4 | | 50.67% | | | C: (16/76) |
| | | | | | 0.64/3.83E−02 |
| | | | | | L: (9/44) |
| | | | | | 0.71/2.65E−02 |
| ACTN4 | 241788_x_at | (I) | 6 | 0.025/4 | |
| Actinin Alpha 4 | | DE/4 | | Stepwise | |
| | | 50% | | | |
| FOXP1 | 223937_at | (I) | 10 | 0.281/0 | |
| Forkhead Box P1 | | DE/4 | | Not Stepwise | |
| | | 54.17% | | | |
| GLRA1 | 207972_at | (I) | 10 | 0.354/0 | Gender/Dx |
| Glycine Receptor | | AP/4 | | Not Stepwise | M-PSYCHOSIS |
| | | 56% | | | C: (29/138) |
| | | | | | 0.6/4.45E−02 |
| | | | | | M-SZA |
| | | | | | C: (16/76) |
| | | | | | 0.64/4.1 6E−02 |
| | | | | | L: (9/44) |
| | | | | | 0.7/3.45E−02 |
| PDE4D | 236610_at | (D) | 10 | 0.418/0 | All |
| Phosphodiesterase 4D | | AP/4 | | Not Stepwise | L: (20/194) |
| | | 60.29% | | | 0.62/4.08E−02 |
| PDE4DIP | 205872_x_at | (I) | 9 | 0.0129/4 | All |
| Phosphodiesterase 4D | | DE/2 | | Stepwise | C: (36/315) |
| Interacting Protein | | 43.06% | | | 0.6/2.87E−02 |
| | | | | | Gender |
| | | | | | Female |
| | | | | | C: (4/30) |
| | | | | | 0.83/1.90E−02 |
| | | | | | Gender/Dx |
| | | | | | F-MDD |
| | | | | | C: (3/28) |
| | | | | | 0.81/4.04E−02 |
| | | | | | M-PSYCHOSIS |
| | | | | | C: (29/138) |
| | | | | | 0.61/3.47E−02 |
| | | | | | M-SZ |
| | | | | | C: (13/62) |
| | | | | | 0.7/1.57E−02 |
| SPON1 | 209436_at | (D) | 10 | 0.248/0 | All |
| Spondin 1 | | DE/4 | | Not Stepwise | L: (20/194) |
| | | 54.05% | | | 0.65/1.60E−02 |
| | | | | | Gender |
| | | | | | Male |
| | | | | | L: (18/176) |
| | | | | | 0.65/2.19E−02 |
| | | | | | Gender/Dx |
| | | | | | M-PSYCHOSIS |
| | | | | | C: (29/138) |
| | | | | | 0.61/3.47E−02 |
| | | | | | L: (17/80) |
| | | | | | 0.67/1.69E−02 |
| | | | | | M-SZA |
| | | | | | L: (9/44) |
| | | | | | 0.72/2.16E−02 |
| SRR | 235677_at | (I) | 10 | 0.712/0 | Gender |
| Serine Racemase | | DE/4 | | Not Stepwise | Female |
| | | 56.94% | | | C: (4/30) |
| | | | | | 0.77/4.38E−02 |
| | | | | | Gender/Dx |
| | | | | | F-MDD |
| | | | | | C: (3/28) |
| | | | | | 0.93/7.89E−03 |
| TCF4 | 212382_at | (I) | 11 | 0.275/2 | Gender |
| Transcription Factor 4 | | DE/2 | | Stepwise | Female |
| | | 33.33% | | | L: (2/18) |
| | | | | | 0.97/1.75E−02 |
| | | | | | Gender/Dx |
| | | | | | F-MDD |
| | | | | | L: (2/18) |
| | | | | | 0.97/1.75E−02 |
| ZBTB20 | 240216_at | (D) | 10 | 0.85/0 | Gender/Dx |
| Zinc Finger And BTB Domain | | AP/4 | | Not Stepwise | M-SZ |
| Containing 20 | | 69.12% | | | L: (8/36) |
| | | | | | 0.71/3.39E−02 |

B. Top Biomarkers for Hallucinations

| | | | | | |
|---|---|---|---|---|---|
| DLG1 | 233869_x_at | (I) | 11 | 3.79E−02/4 | All |
| Discs Large MAG UK | | AP/4 | | Stepwise | L: (29/317) |
| Scaffold Protein 1 | | 55.1% | | | 0.61/2.43E−02 |
| | | | | | Gender |
| | | | | | Male |
| | | | | | L: (29/264) |
| | | | | | 0.61/2.67E−02 |
| | | | | | Gender/Dx |
| | | | | | MalE−SZA |
| | | | | | C: (21/96) |
| | | | | | 0.64/2.64E−02 |
| | | | | | M-BP |
| | | | | | L: (5/107) |
| | | | | | 0.8/1.11E−02 |
| | | | | | M-PSYCHOSIS |
| | | | | | L: (19/101) |
| | | | | | 0.64/3.34E−02 |
| | | | | | M-SZA |
| | | | | | L: (11/57) |
| | | | | | 0.68/3.44E−02 |
| PPP3CB | 215586_at | (I) | 10 | 3.51E−02/4 | All |
| Protein Phosphatase 3 | | AP/4 | | Stepwise | L: (29/317) |
| Catalytic Subunit Beta | | 53.06% | | | 0.6/4.51E−02 |
| | | | | | Gender |
| | | | | | Male |
| | | | | | L: (29/264) |
| | | | | | 0.6/4.68E−02 |
| | | | | | M-PSYCHOSIS |
| | | | | | C: (35/170) |
| | | | | | 0.62/1.30E−02 |
| | | | | | M-SZA |
| | | | | | C: (21/96) |
| | | | | | 0.69/4.4E−03 |
| | | | | | M-BP |
| | | | | | L: (5/107) |
| | | | | | 0.8/1.24E−02 |
| | | | | | M-PSYCHOSIS |
| | | | | | L: (19/101) |
| | | | | | 0.64/2.58E−02 |
| | | | | | M-SZA |
| | | | | | L: (11/57) |
| | | | | | 0.67/4.10E−02 |
| ENPP2 | 210839_s_at | (D) | 8 | 4.13E−01/0 | All |
| Ectonucleotide | | AP/6 | | Not Stepwise | C: (62/513) |
| Pyrophosphatase/Phosphodiesterase | | 94% | | | 0.57/3.98E−02 |
| 2 | | | | | L: (29/317) |
| | | | | | 0.63/9.00E−03 |
| | | | | | Gender |
| | | | | | Male |
| | | | | | L: (29/264) |
| | | | | | 0.63/1.10E−02 |
| | | | | | Gender/Dx |
| | | | | | M-PSYCHOSIS |
| | | | | | C: (35/170) |
| | | | | | 0.61/2.64E−02 |
| | | | | | M-SZA |
| | | | | | C: (21/96) |
| | | | | | 0.67/1.00E−02 |
| | | | | | M-PSYCHOSIS |
| | | | | | L: (19/101) |
| | | | | | 0.68/6.47E−03 |
| ZEB2 | 239296_at | (I) | 10 | 1.13E−01/2 | All |
| Zinc Finger E−Box Binding | | AP/2 | | Stepwise | C: (62/513) |
| Homeobox 2 | | 40.82% | | | 0.58/1.77E−02 |
| | | | | | Gender |
| | | | | | Male |
| | | | | | C: (58/426) |
| | | | | | 0.58/2.30E−02 |
| | | | | | Gender/Dx |
| | | | | | M-PSYCHOSIS |
| | | | | | C: (35/170) |
| | | | | | 0.64/6.71E−03 |
| | | | | | M-SZA |
| | | | | | C: (21/96) |
| | | | | | 0.68/6.30E−03 |
| FNBP1 | 244286_at | (I) | 8 | 2.84E−01/2 | All |
| Formin Binding Protein 1 | | DE/4 | | Stepwise | L: (29/317) |
| | | 50% | | | 0.59/4.93E−02 |
| | | | | | Gender |
| | | | | | Male |
| | | | | | L: (29/264) |
| | | | | | 0.6/4.31E−02 |
| | | | | | M-BP |
| | | | | | L: (5/107) |
| | | | | | 0.83/6.85E−03 |
| | | | | | M-PSYCHOSIS |
| | | | | | C: (35/170) |
| | | | | | 0.6/3.96E−02 |
| | | | | | L: (19/101) |
| | | | | | 0.63/4.04E−02 |
| | | | | | M-SZ |
| | | | | | L: (8/44) |
| | | | | | 0.69/4.42E−02 |
| RTN4 | 243031_at | (I) | 11 | 3.79E−01/2 | All |
| Reticulon 4 | | DE/4 | | Stepwise | L: (29/317) |
| | | 64.81% | | | 0.64/6.96E−03 |
| | | | | | Gender |
| | | | | | Male |
| | | | | | L: (29/264) |
| | | | | | 0.64/6.84E−03 |
| | | | | | Gender/Dx |
| | | | | | M-PTSD |
| | | | | | C: (5/45) |
| | | | | | 0.76/3.02E−02 |
| | | | | | M-BP |
| | | | | | L: (5/107) |
| | | | | | 0.88/1.91E−03 |
| | | | | | M-PSYCHOSIS |
| | | | | | L: (19/101) |
| | | | | | 0.66/1.66E−02 |
| | | | | | M-SZ |
| | | | | | L: (8/44) |
| | | | | | 0.79/5.77E−03 |
| ZNF24/Zinc Finger Protein 24 | 203247_s_at | (I) | 9 | 4.73E−01/2 | All |
| | | DE/4 | | Stepwise | L: (29/317) |
| | | 50% | | | 0.63/1.29E−02 |
| | | | | | Gender |
| | | | | | Males |
| | | | | | L: (29/264) |
| | | | | | 0.63/1.20E−02 |
| | | | | | Gender/Dx |
| | | | | | M-SZA |
| | | | | | C: (21/96) |
| | | | | | 0.64/2.33E−02 |
| | | | | | M-BP |
| | | | | | L: (5/107) |
| | | | | | 0.83/6.57E−03 |
| | | | | | M-SZ |
| | | | | | L: (8/44) |
| | | | | | 0.78/6.86E−03 |
| | | | | | M-PSYCHOSIS |
| | | | | | L: (19/101) |
| | | | | | 0.67/9.26E−03 |
| DST | 215016_x_at | (I) | 10 | 6.44E−02/0 | All |
| Dystonin | | AP/4 | | Not Stepwise | C: (62/513) |
| | | 51.02% | | | 0.57/3.93E−02 |
| | | | | | L: (29/317) |
| | | | | | 0.65/4.61E−03 |
| | | | | | Gender |
| | | | | | Male |
| | | | | | C: (58/426) |
| | | | | | 0.57/4.80E−02 |
| | | | | | L: (29/264) |
| | | | | | 0.65/3.98E−03 |
| | | | | | Gender/Dx |
| | | | | | M-PSYCHOSIS |
| | | | | | C: (35/170) |
| | | | | | 0.61/1.99E−02 |
| | | | | | L: (19/101) |
| | | | | | 0.78/6.13E−05 |
| | | | | | M-SZ |
| | | | | | L: (8/44) |
| | | | | | 0.84/1.43E−03 |
| | | | | | M-SZA |
| | | | | | C: (21/96) |
| | | | | | 0.63/3.10E−02 |
| | | | | | L: (11/57) |
| | | | | | 0.75/5.42E−03 |
| FAT4 | 219427_at | (D) | 8 | 8.09E−01/0 | Gender/Dx |
| FAT Atypical Cadherin 4 | | AP/6 | | Not Stepwise | M-SZ |
| | | 80% | | | L: (8/44) |
| | | | | | 0.69/4.71E−02 |
| ITGAV | 236251_at | (I) | 7 | 3.94E−01/2 | All |
| Integrin Subunit Alpha V | | DE/4 | | Stepwise | L: (29/317) |
| | | 51.85% | | | 0.6/3.33E−02 |
| | | | | | Gender |
| | | | | | Male |
| | | | | | L: (29/264) |
| | | | | | 0.6/3.31E−02 |
| | | | | | Gender/Dx |
| | | | | | M-PTSD |
| | | | | | C: (5/45) |
| | | | | | 0.74/4.15E−02 |
| | | | | | M-BP |
| | | | | | L: (5/107) |
| | | | | | 0.79/1.50E−02 |
| | | | | | M-PSYCHOSIS |
| | | | | | L: (19/101) |
| | | | | | 0.65/1.81E−02 |
| | | | | | M-SZ |
| | | | | | L: (8/44) |
| | | | | | 0.7/4.14E−02 |
| ORMDL1 | 228801_at | (I) | 8 | 5.70E−01/2 | All |
| ORMDL Sphingolipid | | DE/4 | | Stepwise | L: (29/317) |
| Biosynthesis Regulator 1 | | 55.56% | | | 0.63/1.02E−02 |
| | | | | | Gender |
| | | | | | Male |
| | | | | | L: (29/264) |
| | | | | | 0.63/1.07E−02 |
| | | | | | Gender/Dx |
| | | | | | M-BP |
| | | | | | L: (5/107) |
| | | | | | 0.81/9.47E−03 |
| | | | | | M-PSYCHOSIS |
| | | | | | C: (35/170) |
| | | | | | 0.6/3.15E−02 |
| | | | | | L: (19/101) |
| | | | | | 0.71/1.96E−03 |
| | | | | | M-SZ |
| | | | | | L: (8/44) |
| | | | | | 0.84/1.30E−03 |
| ACYP2/Acylphosphatase 2 | 217536_x_at | (I) | 5 | 8.46E−02/2 | Gender/Dx |
| | | DE/6 | | Stepwise | M-PTSD |
| | | 81.48% | | | C: (5/45) |
| | | | | | 0.77/2.56E−02 |
| | | | | | M-PSYCHOSIS |
| | | | | | C: (35/170) |
| | | | | | 0.61/2.59E−02 |
| | | | | | M-SZA |
| | | | | | C: (21/96) |
| | | | | | 0.63/3.04E−02 |
| | | | | | M-BP |
| | | | | | L: (5/107) |
| | | | | | 0.84/5.80E−03 |
| CALM1/Calmodulin 1 | 244869_at | (I) | 8 | 3.40E−02/4 | Gender/Dx |
| | | DE/4 | | Stepwise | M-PTSD |
| | | 77.78% | | | C: (5/45) |
| | | | | | 0.83/8.57E−03 |
| | | | | | M-SZA |
| | | | | | C: (21/96) |
| | | | | | 0.63/3.23E−02 |
| | | | | | M-SZ |
| | | | | | L: (8/44) |
| | | | | | 0.76/1.22E−02 |
| | | | | | M-PSYCHOSIS |
| | | | | | L: (19/101) |
| | | | | | 0.66/1.70E−02 |
| CUL4A/Cullin 4A | 240971_x_at | (I) | 9 | 5.46E−02/2 | Gender/Dx |
| | | DE/2 | | Stepwise | M-PSYCHOSIS |
| | | 44.44% | | | L: (19/101) |
| | | | | | 0.68/8.63E−03 |
| | | | | | M-SZA |
| | | | | | L: (11/57) |
| | | | | | 0.67/3.93E−02 |
| DHX36/DEAH-Box Helicase 36 | 1559039_at | (I) | 5 | 3.55E−02/4 | All |
| | | AP/4 | | Stepwise | L: (29/317) |
| | | 57.14% | | | 0.62/1.44E−02 |
| | | | | | Gender |
| | | | | | Males |
| | | | | | L: (29/264) |
| | | | | | 0.63/8.95E−03 |
| | | | | | Gender/Dx |
| | | | | | M-PSYCHOSIS |
| | | | | | C: (35/170) |
| | | | | | 0.6/3.89E−02 |
| | | | | | L: (19/101) |
| | | | | | 0.67/1.22E−02 |
| | | | | | M-PTSD |
| | | | | | C: (5/45) |
| | | | | | 0.73/4.83E−02 |
| | | | | | M-SZA |
| | | | | | C: (21/96) |
| | | | | | 0.64/2.92E−02 |
| | | | | | L: (11/57) |
| | | | | | 0.68/3.60E−02 |
| GNAS/GNAS Complex Locus | 211858_x_at | (D) | 10 | 2.52E−01/0 | All |
| | | DE/4 | | Not Stepwise | L: (29/317) |
| | | 53.85% | | | 0.6/4.31E−02 |
| | | | | | Gender |
| | | | | | Males |
| | | | | | L: (29/264) |
| | | | | | 0.6/4.55E−02 |
| HDAC8/Histone Deacetylase 8 | 223908_at | (I) | 6 | 1.11E−01/2 | All |
| | | DE/6 | | Stepwise | L: (29/317) |
| | | 81.48% | | | 0.63/1.19E−02 |
| | | | | | Gender |
| | | | | | Females |
| | | | | | C: (4/87) |
| | | | | | 0.79/2.71E−02 |
| | | | | | Males |
| | | | | | L: (29/264) |
| | | | | | 0.63/1.21E−02 |
| | | | | | Gender/Dx |
| | | | | | M-SZ |
| | | | | | L: (8/44) |
| | | | | | 0.89/2.92E−04 |
| | | | | | M-PSYCHOSIS |
| | | | | | L: (19/101) |
| | | | | | 0.72/1.25E−03 |
| MAB21L1/Mab-21 Like 1 | 206163_at | (D) | 10 | 3.06E−01/2 | All |
| | | DE/2 | | Stepwise | L: (29/317) |
| | | 36.92% | | | 0.62/1.62E−02 |
| | | | | | Gender |
| | | | | | Males |
| | | | | | C: (58/426) |
| | | | | | 0.57/4.80E−02 |
| | | | | | L: (29/264) |
| | | | | | 0.62/1.64E−02 |
| | | | | | M-PSYCHOSIS |
| | | | | | C: (35/170) |
| | | | | | 0.64/5.96E−03 |
| | | | | | L: (19/101) |
| | | | | | 0.68/8.23E−03 |
| | | | | | M-SZ |
| | | | | | C: (14/74) |
| | | | | | 0.68/1.74E−02 |
| | | | | | L: (8/44) |
| | | | | | 0.77/8.11E−03 |
| ZBTB20/Zinc Finger And BTB | 235308_at | (I) | 6 | 3.90E−02/4 | All |
| Domain Containing 20 | | DE/4 | | Stepwise | L: (29/317) |
| | | 51.85% | | | 0.61/2.37E−02 |
| | | | | | Gender |
| | | | | | Males |
| | | | | | L: (29/264) |
| | | | | | 0.61/2.37E−02 |
| | | | | | Gender/Dx |
| | | | | | M-PSYCHOSIS |
| | | | | | C: (35/170) |
| | | | | | 0.61/2.05E−02 |
| | | | | | L: (19/101) |
| | | | | | 0.69/4.81E−03 |
| | | | | | M-SZ |
| | | | | | C: (21/96) |
| | | | | | 0.65/1.93E−02 |
| | | | | | L: (8/44) |
| | | | | | 0.75/1.42E−02 |
| ZNF24/Zinc Finger Protein 24 | 242210_at | (I) | 9 | 9.54E−01/0 | All |
| | | DE/4 | | Not Stepwise | L: (29/317) |
| | | 53.7% | | | 0.62/1.52E−02 |
| | | | | | Gender |
| | | | | | Males |
| | | | | | L: (29/264) |
| | | | | | 0.62/1.45E−02 |
| | | | | | Gender/Dx |
| | | | | | M-BP |
| | | | | | L: (5/107) |
| | | | | | 0.87/2.76E−03 |
| | | | | | M-PSYCHOSIS |
| | | | | | L: (19/101) |
| | | | | | 0.63/3.53E−02 |
| | | | | | M-SZ |
| | | | | | L: (8/44) |
| | | | | | 0.69/4.71E−02 |
| CTNND1 | 1557944_s_at | (D) | 9 | 8.94E−02/2 | Gender/Dx |
| Catenin Delta 1 | | DE/2 | | Stepwise | M-PTSD |
| | | 33.85 | | | C: (5/45) |
| | | | | | 0.8/1.66E−02 |
| | | | | | M-BP |
| | | | | | L: (5/107) |
| | | | | | 0.73/4.21E−02 |

| | | | | | |
|---|---|---|---|---|---|
| | | Step 4 | | | |
| | | Best Significant | | | |
| | | Predictions of All | | | |
| | | Future Hosp for | | | |
| | | Delusions OR/OR | | | |
| | Step 4 | p-value Updated | | | |
| | Significant Prediction | 1-tailed p-value | | Step 6 | CFE |
| | of First Year Hosp for | and added new data | | Drugs that | Polyevidence |
| | Delusions All Gender | for genes that has | Step 5 | Modulate the | Score for |
| | Best in Individualized | now signif p-value | Other Psychiatric | Biomarker in | Involvement |
| | Gender/Dx ROC AUC/p-value | 3 pts All | and Related | Same Direction | in Delusions |
| | 3 pts All | 2pts Gender | Disorders | as High | (Based on |
| Gene Symbol/Gene Name | 2 pts Gender | 1 pts Gender/Dx | Evidence | Delusions | Steps 1-4) |

A. Top Biomarkers for Delusions.

| | | | | | |
|---|---|---|---|---|---|
| AUTS 2 | All | Gender | Alzheimer's | Lithium | 24 |
| Activator Of Transcription And | L: (7/158) | Female | Disease | Citalopram | |
| Developmental Regulator AUTS 2 | 0.74/1.58E−02 | C: (12/39) | Aging | | |
| | Gender/Dx | 1.43/1.64E−02 | Alcohol | | |
| | M-BP | Gender/Dx | ASD | | |
| | L: (1/90) | F-MDD | BP | | |
| | 0.99/4.70E−02 | C: (2/25) | PTSD | | |
| | | 1.74/2.61E−02 | Cannabis | | |
| | | F-SZA | | | |
| | | C: (6/9) | | | |
| | | 4.45/2.21E−02 | | | |
| | | M-SZ | | | |
| | | L: (9/34) | | | |
| | | 2.7/2.69E−02 | | | |
| NR4A2 | All | All | Alcohol | | 23 |
| Nuclear Receptor Subfamily 4 | C: (15/254) | C: (80/292) | MDD | | |
| Group A Member 2 | 0.69/5.87E−03 | 1.19/2.88E−02 | Alzheimer's | | |
| | L: (7/158) | Gender | Disease | | |
| | 0.79/5.29E−03 | Female | Aging | | |
| | Gender | C: (12/39) | PTSD | | |
| | Female | 1.96/8.64E−03 | | | |
| | C: (4/11) | Gender/Dx | | | |
| | 0.96/7.01E−03 | F-PSYCHOSIS | | | |
| | L: (2/6) | C: (7/11) | | | |
| | 1/3.20E−02 | 3.61/1.24E−02 | | | |
| | Male | L: (4/6) | | | |
| | L: (5/152) | 7.9/4.92E−02 | | | |
| | 0.72/4.66E−02 | F-SZA | | | |
| | Gender/Dx | C: (6/9) | | | |
| | M-BP | 3.42/2.36E−02 | | | |
| | C: (2/135) | L: (4/6) | | | |
| | 0.97/1.09E−02 | 7.9/4.92E−02 | | | |
| | L: (1/90) | | | | |
| | 1/4.34E−02 | | | | |
| | F-PSYCHOSIS | | | | |
| | C: (4/ll) | | | | |
| | 0.96/7.01E−03 | | | | |
| | L: (2/6) | | | | |
| | 1/3.20E−02 | | | | |
| | F-SZA | | | | |
| | C: (3/9) | | | | |
| | 0.94/1.94E−02 | | | | |
| | L: (2/6) | | | | |
| | 1/3.20E−02 | | | | |
| CHD 9 | All | All | | | 22 |
| Chromodomain Helicase DNA | L: (7/158) | C: (80/292) | | | |
| Binding Protein 9 | 0.69/4.51E−02 | 1.2/2.40E−02 | | | |
| | Gender | L: (43/176) | | | |
| | Female | 1.48/3.96E−02 | | | |
| | L: (2/6) | Gender | | | |
| | 1/3.20E−02 | Male | | | |
| | Gender/Dx | C: (68/253) | | | |
| | M-BP | 1.19/3.72E−02 | | | |
| | C: (2/135) | Gender/Dx | | | |
| | 0.92/1.98E−02 | F-MDD | | | |
| | L: (1/90) | C: (2/25) | | | |
| | 0.99/4.70E−02 | 2.19/2.58E−02 | | | |
| | F-PSYCHOSIS | M-PSYCHOSIS | | | |
| | L: (2/6) | C: (39/124) | | | |
| | 1/3.20E−02 | 1.3/2.76E−02 | | | |
| | F-SZA | M-SZA | | | |
| | C: (3/9) | C: (22/66) | | | |
| | 0.89/3.54E−02 | 1.4/2.19E−02 | | | |
| | L: (2/6) | L: (11/38) | | | |
| | 1/3.20E−02 | 2.99/5.69E−04 | | | |
| IL6ST | All | All | Male | Fluoxetine | 22 |
| Interleukin 6 Signal Transducer | C: (15/254) | C: (80/292) | MDD | | |
| | 0.63/4.98E−02 | 1.19/4.53E−02 | BP | | |
| | Gender/Dx | Gender | Later-Life | | |
| | M-BP | Male | Depression | | |
| | C: (2/135) | C: (68/253) | (LLD) | | |
| | 0.89/3.03E−02 | 1.24/2.47E−02 | Stress | | |
| | | Gender/Dx | | | |
| | | M-BP | | | |
| | | C: (24/122) | | | |
| | | 1.62/2.67E−03 | | | |
| | | L: (15/78) | | | |
| | | 1.94/2.05E−02 | | | |
| | | M-SZA | | | |
| | | C: (22/66) | | | |
| | | 1.33/4.85E−02 | | | |
| MACROD2 | Gender/Dx | All | | | 21 |
| Mono-ADP Ribosylhydrolase 2 | M-BP | C: (80/292) | | | |
| | C: (2/135) | 1.22/1.66E−02 | | | |
| | 0.86/3.86E−02 | Gender | | | |
| | | Male | | | |
| | | C: (68/253) | | | |
| | | 1.23/1.90E−02 | | | |
| | | Gender/Dx | | | |
| | | F-MDD | | | |
| | | C: (2/25) | | | |
| | | 2.2/3.64E−02 | | | |
| | | M-SZA | | | |
| | | C: (22/66) | | | |
| | | 1.32/4.97E−02 | | | |
| PDP1 | Gender | M-SZA | | Omega-3 | 21 |
| Pyruvate Dehyrogenase Phosphatase | Male | C: (22/66) | | fatty acids | |
| Catalytic Subunit 1 | C: (11/243) | 1.49/2.02E−02 | | Fluoxetine | |
| | 0.68/2.22E−02 | | | Lithium | |
| | Gender/Dx | | | CPI-613 | |
| | M-BP | | | | |
| | C: (2/135) | | | | |
| | 1/7.71E−03 | | | | |
| RORA RAR | Gender/Dx | All | Suicide | Risperidone | 21 |
| Related Orphan Receptor A | M-BP | C: (80/292) | Aging | Fluoxetine | |
| | C: (2/135) | 1.3/4.60E−03 | ASD | Lithium | |
| | 0.9/2.57E−02 | Gender | | | |
| | L: (1/90) | Male | | | |
| | 1/4.34E−02 | C: (68/253) | | | |
| | | 1.35/1.99E−03 | | | |
| | | L: (38/154) | | | |
| | | 1.44/2.73E−02 | | | |
| | | M-BP | | | |
| | | C: (24/122) | | | |
| | | 2.08/1.21E−05 | | | |
| | | L: (15/78) | | | |
| | | 2.72/8.99E−04 | | | |
| | | M-SZA | | | |
| | | L: (11/38) | | | |
| | | 2.06/1.28E−02 | | | |
| FOXP1 | Gender | All | Alcohol | Ketamine | 20 |
| Forkhead Box P1 | Male | C: (80/292) | | Omega-3 | |
| | C: (11/243) | 1.19/4.73E−02 | | fatty acids | |
| | 0.69/1.88E−02 | Gender/Dx | | | |
| | Gender/Dx | M-BP | | | |
| | M-BP | C: (24/122) | | | |
| | C: (2/135) | 1.49/2.38E−02 | | | |
| | 0.86/4.02E−02 | L: (15/78) | | | |
| | L: (1/90) | 1.85/2.57E−02 | | | |
| | 0.99/4.70E−02 | | | | |
| | M-PSYCHOSIS | | | | |
| | C: (6/101) | | | | |
| | 0.71/4.37E−02 | | | | |
| FOXP1 | Gender/Dx | Gender/Dx | MDD | Omega-3 | 20 |
| Forkhead Box P1 | M-SZ | F-PSYCHOSIS | Aging | fatty acids | |
| | L: (2/36) | C: (7/11) | Circadian | Clozapine | |
| | 0.87/4.21E−02 | 2.53/2.52E−02 | abnormalities | | |
| | | | Cannabis | | |
| | | | BP | | |
| | | | Stress | | |
| GNAS | All | Gender | | Risperidone | 20 |
| GNAS Complex Locus | C: (15/254) | Male | | Valproic acid | |
| | 0.66/2.17E−02 | C: (68/253) | | Clozapine | |
| | Gender | 1.2/4.69E−02 | | | |
| | Male | L: (38/154) | | | |
| | C: (11/243) | 1.32/4.45E−02 | | | |
| | 0.67/2.90E−02 | Gender/Dx | | | |
| | Gender/Dx | M-BP | | | |
| | M-BP | C: (24/122) | | | |
| | C: (2/135) | 1.35/4.86E−02 | | | |
| | 0.97/1.14E−02 | L: (15/78) | | | |
| | L: (1/90) | 1.77/1.00E−02 | | | |
| | 1/4.34E−02 | M-SZA | | | |
| | | C: (22/66) | | | |
| | | 1.49/1.32E−02 | | | |
| XRCC6 | Gender/Dx | All | Social | | 20 |
| X-Ray Repair Cross | M-BP | C: (80/292) | Isolation | | |
| Complementing 6 | C: (2/135) | 1.4/3.02E−04 | Aging | | |
| | 1/8.11E−03 | Gender | Suicide | | |
| | L: (1/90) | Male | Completers | | |
| | 1/4.34E−02 | C: (68/253) | Depression | | |
| | | 1.45/9.00E−05 | BP | | |
| | | Gender/Dx | | | |
| | | M-BP | | | |
| | | C: (24/122) | | | |
| | | 1.72/1.41E−04 | | | |
| | | L: (15/78) | | | |
| | | 2.34/1.40E−02 | | | |
| | | M-PSYCHOSIS | | | |
| | | C: (39/124) | | | |
| | | 1.38/9.06E−03 | | | |
| | | M-SZA | | | |
| | | C: (22/66) | | | |
| | | 1.5/6.88E−03 | | | |
| ZBTB20 | | Gender/Dx | Social | Risperidone | 20 |
| Zinc Finger And BTB Domain | | F-SZA | Isolation | Fluoxetine | |
| Containing 20 | | C: (6/9) | Aging | | |
| | | 5.31/4.76E−02 | Suicide | | |
| | | | Completers | | |
| | | | Depression | | |
| | | | BP | | |
| ACSL4 | | All | MDD | Lithium | 19 |
| Acyl-CoA Synthetase Long | | C: (80/292) | Suicide | Omega-3 | |
| Chain Family Member 4 | | 1.25/1.44E−02 | Completers | fatty acids | |
| | | Gender | BP | | |
| | | Male | Huntington's | | |
| | | C: (68/253) | Disease | | |
| | | 1.23/3.30E−02 | Parkinson | | |
| | | Gender/Dx | Stress | | |
| | | M-PSYCHOSIS | ASD | | |
| | | C: (39/124) | | | |
| | | 1.35/1.88E−02 | | | |
| | | L: (20/72) | | | |
| | | 1.93/1.34E−02 | | | |
| | | M-SZ | | | |
| | | C: (17/58) | | | |
| | | 1.51/1.27E−02 | | | |
| | | L: (9/34) | | | |
| | | 4.14/1.59E−03 | | | |
| ACTN4 | Gender/Dx | All | | | 19 |
| Actinin Alpha 4 | M-BP | C: (80/292) | | | |
| | L: (1/90) | 1.2/1.37E−02 | | | |
| | 0.99/4.70E−02 | Gender | | | |
| | | Male | | | |
| | | C: (68/253) | | | |
| | | 1.21/1.16E−02 | | | |
| | | Gender/Dx | | | |
| | | M-BP | | | |
| | | C: (24/122) | | | |
| | | 1.26/2.15E−02 | | | |
| FOXP1 | Gender/Dx | All | Alcohol | Ketamine | 19 |
| Forkhead Box P1 | M-BP | C: (80/292) | | Omega-3 | |
| | C: (2/135) | 1.17/4.07E−02 | | fatty acids | |
| | 0.98/1.04E−02 | Gender | | | |
| | L: (1/90) | Male | | | |
| | 1/4.34E−02 | C: (68/253) | | | |
| | | 1.17/4.29E−02 | | | |
| | | M-BP | | | |
| | | C: (24/122) | | | |
| | | 1.39/2.14E−03 | | | |
| | | L: (15/78) | | | |
| | | 2.15/2.85E−02 | | | |
| | | M-SZA | | | |
| | | L: (11/38) | | | |
| | | 1.84/2.54E−02 | | | |
| GLRA1 | | All | Aging | Clozapine | 19 |
| Glycine Receptor | | C: (80/292) | MDD | Haloperidol | |
| | | 1.2/3.44E−02 | Suicide | | |
| | | | BP | | |
| | | | Anxiety | | |
| | | | PTSD | | |
| PDE4D | M-PSYCH | | MDD | TCA | 19 |
| Phosphodiesterase 4D | C: (3/7) | | Autism | Olanzapine | |
| | 1/1.69E−02 | | Alzheimer's | Risperidone | |
| | M-PSYCHOSIS | | Disease | Clozapine | |
| | L: (2/58) | | Social | | |
| | 0.95/1.66E−02 | | Isolation | | |
| | M-SZ | | Chronic | | |
| | L: (2/36) | | Stress | | |
| | 0.96/1.61E−02 | | Longevity | | |
| PDE4DIP | | | | Clozapine | 19 |
| Phosphodiesterase 4D | | | | | |
| Interacting Protein | | | | | |
| SPON1 | | Gender/Dx | MDD | Clozapine | 19 |
| Spondin 1 | | F-PSYCHOSIS | Suicide | | |
| | | C: (7/11) | | | |
| | | 2.34/4.39E−02 | | | |
| | | M-SZ | | | |
| | | C: (17/58) | | | |
| | | 1.63/3.84E−02 | | | |
| SRR | Gender | Gender/Dx | | | 19 |
| Serine Racemase | Male | F-MDD | | | |
| | L: (5/152) | C: (2/25) | | | |
| | 0.77/1.92E−02 | 19.59/4.90E−02 | | | |
| | Gender/Dx | | | | |
| | M-BP | | | | |
| | C: (2/135) | | | | |
| | 0.98/9.40E−03 | | | | |
| | M-PSYCHOSIS | | | | |
| | L: (2/58) | | | | |
| | 0.85/4.83E−02 | | | | |
| | M-SZ | | | | |
| | L: (2/36) | | | | |
| | 0.87/4.21E−02 | | | | |
| TCF4 | Gender/Dx | Gender/Dx | Alzheimer's | Dexamethasone | 19 |
| Transcription Factor 4 | M-BP | M-BP | Disease | Valproate | |
| | C: (2/135) | C: (24/122) | BP | CBD | |
| | 0.91/2.26E−02 | 1.41/1.46E−02 | Males | | |
| | | | Suicide | | |
| | | | Completers | | |
| | | | Alcohol | | |
| ZBTB20 | Gender | Gender | Social | Risperidone | 19 |
| Zinc Finger And BTB Domain | Female | Female | Isolation | Fluoxetine | |
| Containing 20 | C: (4/11) | C: (12/39) | Aging | | |
| | 0.82/4.45E−02 | 1.84/2.69E−02 | Suicide | | |
| | Gender/Dx | Gender/Dx | Completers | | |
| | F-PSYCHOSIS | F-PSYCHOSIS | Depression | | |
| | C: (4/11) | C: (7/11) | BP | | |
| | 0.82/4.45E−02 | 5.31/1.27E−02 | | | |
| | M-PSYCH | F-SZA | | | |
| | C: (3/7) | C: (6/9) | | | |
| | 1/1.69E−02 | 4.69/2.76E−02 | | | |
| | M-SZ | | | | |
| | L: (2/36) | | | | |
| | 0.9/3.11E−02 | | | | |

B. Top Biomarkers for Hallucinations

| | | | | | |
|---|---|---|---|---|---|
| DLG1 | All | All | Suicide | Clozapine | 31 |
| Discs Large MAG UK | C: (22/437) | C: (108/442) | Stress | | |
| Scaffold Protein 1 | 0.61/4.18E−02 | 1.16/1.59E−02 | Pain | | |
| | Gender | L: (60/270) | Anxiety | | |
| | Female | 1.43/8.39E−03 | Addiction | | |
| | C: (4/48) | Gender | | | |
| | 0.84/1.39E−02 | Male | | | |
| | Male | C: (99/392) | | | |
| | L: (10/243) | 1.14/4.07E−02 | | | |
| | 0.68/3.01E−02 | L: (57/239) | | | |
| | Gender/Dx | 1.4/1.40E−02 | | | |
| | M-PTSD | Gender/Dx | | | |
| | C: (4/39) | M-MDD | | | |
| | 0.76/4.34E−02 | C: (9/78) | | | |
| | M-MDD | 1.75/1.16E−02 | | | |
| | L: (2/34) | M-PTSD | | | |
| | 0.94/2.02E−02 | C: (10/39) | | | |
| | | 2.07/6.48E−04 | | | |
| | | M-PTSD | | | |
| | | L: (7/25) | | | |
| | | 3.93/2.31E−03 | | | |
| PPP3CB | All | All | Addictions | Clozapine | 30 |
| Protein Phosphatase 3 | C: (22/437) | C: (108/442) | Aging | | |
| Catalytic Subunit Beta | 0.64/1.47E−02 | 1.2/7.59E−03 | Alzheimer's | | |
| | Gender | Gender | Disease | | |
| | Female | Female | Suicide | | |
| | C: (4/48) | C: (9/50) | Depression | | |
| | 0.88/6.22E−03 | 2.31/1.10E−02 | Bipolar | | |
| | F-BP | Male | Disorder | | |
| | C: (2/24) | C: (99/392) | PTSD | | |
| | 0.89/3.79E−02 | 1.15/4.26E−02 | Pain | | |
| | M-MDD | Gender/Dx | ASD | | |
| | C: (3/56) | M-MDD | | | |
| | 0.82/3.31E−02 | C: (9/78) | | | |
| | | 1.59/4.17E−02 | | | |
| | | M-PTSD | | | |
| | | C: (10/39) | | | |
| | | 1.84/1.01E−02 | | | |
| ENPP2 | All | All | MDD | Fluoxetine | 26 |
| Ectonucleotide | C: (22/437) | L: (60/270) | Addictions | Valproic acid | |
| Pyrophosphatase/Phosphodiesterase | 0.65/8.18E−03 | 1.54/1.51E−02 | Alzheimer's | | |
| 2 | Gender | Gender | Disease | | |
| | Female | Male | Aging | | |
| | C: (18/389) | L: (57/239) | Stress | | |
| | 0.66/9.16E−03 | 1.61/1.00E−02 | Pain | | |
| | Male | Gender/Dx | | | |
| | L: (10/243) | M-BP | | | |
| | 0.68/2.60E−02 | L: (13/82) | | | |
| | Gender/Dx | 2.09/3.92E−02 | | | |
| | M-PSYCHOSIS | M-PSYCHOSIS | | | |
| | C: (8/153) | C: (54/147) | | | |
| | 0.72/1.76E−02 | 1.5/2.80E−02 | | | |
| | M-SZ | L: (31/86) | | | |
| | C: (4/72) | 1.67/3.07E−02 | | | |
| | 0.76/3.84E−02 | M-SZ | | | |
| | L: (3/43) | C: (37/81) | | | |
| | 0.83/2.83E−02 | 1.98/2.69E−02 | | | |
| | | L: (10/39) | | | |
| | | 3.89/2.49E−02 | | | |
| ZEB2 | All | All | Dementia | Clozapine, | 26 |
| Zinc Finger E−Box Binding | C: (22/437) | C: (108/442) | Depression | CelastrolOmega-3 | |
| Homeobox 2 | 0.64/1.28E−02 | 1.24/1.50E−03 | Aging | fatty acids | |
| | Gender | FemalE−BP | Stress | | |
| | Female | C: (2/24) | Suicide | | |
| | C: (4/48) | 2.05/4.00E−02 | | | |
| | 0.83/1.52E−02 | Gender | | | |
| | Gender/Dx | Female | | | |
| | M-PTSD | C: (9/50) | | | |
| | C: (4/39) | 1.99/6.11E−03 | | | |
| | 0.81/2.08E−02 | Male | | | |
| | L: (3/25) | C: (99/392) | | | |
| | 0.88/1.83E−02 | 1.19/1.14E−02 | | | |
| | | Gender/Dx | | | |
| | | M-PSYCHOSIS | | | |
| | | C: (54/147) | | | |
| | | 1.21/4.76E−02 | | | |
| | | M-PTSD | | | |
| | | C: (10/39) | | | |
| | | 2.51/9.48E−04 | | | |
| | | L: (7/25) | | | |
| | | 5.65/3.94E−03 | | | |
| FNBP1 | Gender | All | Depression | Olanzapine | 24 |
| Formin Binding Protein 1 | Female | C: (108/442) | Alzheimer's | | |
| | C: (4/48) | 1.14/3.82E−02 | Disease, | | |
| | 0.81/2.20E−02 | M-PTSD | Aging | | |
| | Gender/Dx | C: (10/39) | ASD | | |
| | M-PTSD | 3.42/8.70E−05 | Pain | | |
| | C: (4/39) | L: (7/25) | Suicide | | |
| | 0.76/4.78E−02 | 4.4/4.44E−03 | Stress | | |
| | M-SZ | | Addictions | | |
| | L: (3/43) | | | | |
| | 0.79/4.76E−02 | | | | |
| RTN4 | Gender | M-PTSD | Depression | Valproate | 24 |
| Reticulon 4 | Male | C: (10/39) | Bipolar | Omega-3 | |
| | L: (10/243) | 2.82/1.74E−04 | Disorder | fatty acids | |
| | 0.68/2.57E−02 | L: (7/25) | Stress | | |
| | M-PTSD | 3.68/8.69E−03 | Suicide | | |
| | C: (4/39) | | Dementia, | | |
| | 0.89/6.21E−03 | | Pain | | |
| | L: (3/25) | | Addictions | | |
| | 0.82/3.95E−02 | | | | |
| ZNF24/Zinc Finger Protein 24 | | All | BP | | 23 |
| | | C: (108/442) | Alzheimer's | | |
| | | 1.19/1.63E−02 | Disease | | |
| | | Gender | Aggression | | |
| | | Female | Stress | | |
| | | C: (9/50) | | | |
| | | 1.69/3.75E−02 | | | |
| | | L: (3/31) | | | |
| | | 3.11/4.68E−02 | | | |
| | | Male | | | |
| | | C: (99/392) | | | |
| | | 1.16/4.76E−02 | | | |
| | | Gender/Dx | | | |
| | | M-PTSD | | | |
| | | L: (7/25) | | | |
| | | 3.75/1.99E−02 | | | |
| | | C: (10/39) | | | |
| | | 3.09/3.18E−04 | | | |
| DST | Gender/Dx | All | Depression | Risperidone | 23 |
| Dystonin | M-MDD | C: (108/442) | Alzheimer's | | |
| | L: (2/34) | 1.2/1.84E−02 | Disease | | |
| | 0.94/2.02E−02 | L: (60/270) | ASD | | |
| | | 1.4/5.42E−03 | Pain | | |
| | | Gender | Suicide | | |
| | | Male | | | |
| | | C: (99/392) | | | |
| | | 1.22/1.22E−02 | | | |
| | | L: (57/239) | | | |
| | | 1.41/4.57E−03 | | | |
| | | Gender/Dx | | | |
| | | M-MDD | | | |
| | | C: (9/78) | | | |
| | | 2.39/2.28E−05 | | | |
| | | L: (6/46) | | | |
| | | 6.86/2.17E−04 | | | |
| FAT4 | All | All | Aging | Citalopram | 23 |
| FAT Atypical Cadherin 4 | C: (22/437) | C: (108/442) | Suicide | | |
| | 0.61/3.54E−02 | 1.21/3.11E−02 | Stress | | |
| | | L: (60/270) | | | |
| | | 1.91/2.18E−04 | | | |
| | | Gender | | | |
| | | Male | | | |
| | | C: (99/392) | | | |
| | | 1.2/4.38E−02 | | | |
| | | L: (57/239) | | | |
| | | 1.77/1.62E−03 | | | |
| | | M-PTSD | | | |
| | | C: (10/39) | | | |
| | | 2.02/3.31E−02 | | | |
| | | L: (7/25) | | | |
| | | 3.23/3.69E−02 | | | |
| ITGAV | Gender | All | ASD | Valproate | 23 |
| Integrin Subunit Alpha V | Female | C: (108/442) | Alzheimer's | | |
| | C: (4/48) | 1.14/4.95E−02 | Disease | | |
| | 0.79/2.86E−02 | L: (60/270) | Depression | | |
| | Gender/Dx | 1.28/4.90E−02 | PTSD | | |
| | M-PTSD | Gender/Dx | Addictions | | |
| | C: (4/39) | M-PTSD | Aging | | |
| | 0.86/1.03E−02 | C: (10/39) | | | |
| | | 3.08/4.74E−04 | | | |
| | | M-MDD | | | |
| | | L: (6/46) | | | |
| | | 1.49/4.76E−02 | | | |
| ORMDL1 | All | Gender/Dx | Stress | | 23 |
| ORMDL Sphingolipid | C: (22/437) | M-PTSD | Depression | | |
| Biosynthesis Regulator 1 | 0.61/4.16E−02 | C: (10/39) | Aging | | |
| | Gender | 2.17/1.59E−03 | PTSD | | |
| | Female | L: (7/25) | Pain | | |
| | C: (4/48) | 2.72/2.96E−02 | | | |
| | 0.82/1.67E−02 | | | | |
| ACYP2/Acylphosphatase 2 | All | All | Alzheimer's | | 22 |
| | C: (22/437) | C: (108/442) | Disease | | |
| | 0.65/9.03E−03 | 1.14/3.10E−02 | Progression | | |
| | Gender | All | Aging | | |
| | Females | L: (60/270) | Stress | | |
| | C: (4/48) | 1.33/2.08E−02 | | | |
| | 0.81/2.20E−02 | Gender | | | |
| | Males | Male | | | |
| | L: (10/243) | L: (57/239) | | | |
| | 0.65/4.86E−02 | 1.29/3.81E−02 | | | |
| | Gender/Dx | Gender/Dx | | | |
| | M-PTSD | M-PTSD | | | |
| | C: (4/39) | C: (10/39) | | | |
| | 0.88/7.08E−03 | 1.77/5.50E−03 | | | |
| | M-SZ | L: (7/25) | | | |
| | L: (3/43) | 2.68/6.06E−03 | | | |
| | 0.89/1.25E−02 | M-PSYCHOSIS | | | |
| | | L: (31/86) | | | |
| | | 1.49/2.26E−02 | | | |
| | | M-SZA | | | |
| | | L: (21/47) | | | |
| | | 1.49/3.69E−02 | | | |
| CALM1/Calmodulin 1 | All | All | Suicide | | 22 |
| | C: (22/437) | C: (108/442) | Completers | | |
| | 0.63/2.18E−02 | 1.16/1.66E−02 | Male | | |
| | Gender | L: (60/270) | MDD | | |
| | Females | 1.27/3.46E−02 | Alzheimer's | | |
| | C: (4/48) | Gender | Disease | | |
| | 0.78/3.38E−02 | Male | Childhood | | |
| | F-MDD | C: (99/392) | Trauma | | |
| | C: (1/21) | 1.15/2.71E−02 | ASD | | |
| | 1/4.93E−02 | Gender/Dx | Parkinson | | |
| | Males | F-MDD | Aging | | |
| | L: (10/243) | C: (4/23) | Alcohol | | |
| | 0.67/3.62E−02 | 2.19/4.72E−02 | | | |
| | Gender/Dx | M-MDD | | | |
| | M-PTSD | C: (9/78) | | | |
| | C: (4/39) | 1.62/6.95E−03 | | | |
| | 0.89/5.45E−03 | M-PTSD | | | |
| | M-MDD | C: (10/39) | | | |
| | L: (2/34) | 1.58/1.14E−02 | | | |
| | 0.86/4.61E−02 | M-PSYCHOSIS | | | |
| | | L: (31/86) | | | |
| | | 1.34/4.05E−02 | | | |
| | | All | | | |
| CUL4A/Cullin 4A | | All | Alzheimer's | Clozapine | 22 |
| | | C: (108/442) | Disease | Omega-3 | |
| | | 1.14/3.76E−02 | Aging | fatty acids | |
| | | L: (60/270) | Stress | | |
| | | 1.37/9.52E−03 | | | |
| | | Gender | | | |
| | | Male | | | |
| | | L: (57/239) | | | |
| | | 1.36/1.39E−02 | | | |
| | | M-MDD | | | |
| | | C: (9/78) | | | |
| | | 1.54/1.92E−02 | | | |
| | | M-PTSD | | | |
| | | C: (10/39) | | | |
| | | 2.44/4.88E−04 | | | |
| | | L: (7/25) | | | |
| | | 5.03/9.29E−04 | | | |
| DHX36/DEAH-Box Helicase 36 | All | Gender/Dx | MDD | Risperidone | 22 |
| | C: (22/437) | M-PTSD | | | |
| | 0.61/4.30E−02 | C: (10/39) | | | |
| | | 2.66/1.46E−03 | | | |
| | | L: (7/25) | | | |
| | | 2.45/2.61E−02 | | | |
| GNAS/GNAS Complex Locus | Gender | Gender | BP | Valproic acid | 22 |
| | Females | Female | Alzheimer's | Antipsychotics | |
| | C: (4/48) | C: (9/50) | Disease | Clozapine | |
| | 0.82/1.84E−02 | 2.54/6.84E−03 | Suicide | | |
| | Gender/Dx | Gender/Dx | Childhood | | |
| | M-MDD | F-MDD | Trauma | | |
| | L: (2/34) | C: (4/23) | Aging | | |
| | 0.86/4.61E−02 | 3.93/1.36E−02 | Cocaine | | |
| | | M-MDD | Anxiety | | |
| | | C: (9/78) | Alcohol | | |
| | | 2.24/1.98E−02 | | | |
| | | L: (6/46) | | | |
| | | 4.23/5.03E−03 | | | |
| HDAC8/Histone Deacetylase 8 | | All | Panic | Lithium | 22 |
| | | C: (108/442) | Disorder | | |
| | | 1.16/4.46E−02 | MDD | | |
| | | Gender | Stress | | |
| | | Male | | | |
| | | C: (99/392) | | | |
| | | 1.17/3.36E−02 | | | |
| | | Gender/Dx | | | |
| | | M-MDD | | | |
| | | C: (9/78) | | | |
| | | 2.38/1.17E−05 | | | |
| | | L: (6/46) | | | |
| | | 3.58/3.55E−04 | | | |
| | | M-SZ | | | |
| | | C: (17/66) | | | |
| | | 1.42/1.65E−02 | | | |
| MAB21L1/Mab-21 Like 1 | | All | MDD | Escitalopram | 22 |
| | | C: (108/442) | Stress | (SSRI) | |
| | | 1.24/2.32E−02 | | Clozapine | |
| | | Gender | | | |
| | | Male | | | |
| | | C: (99/392) | | | |
| | | 1.3/1.08E−02 | | | |
| | | M-MDD | | | |
| | | C: (9/78) | | | |
| | | 2.02/3.15E−02 | | | |
| | | M-BP | | | |
| | | C: (26/128) | | | |
| | | 1.6/1.72E−02 | | | |
| ZBTB20/Zinc Finger And BTB | | All | Alcohol | Risperidone | 22 |
| Domain Containing 20 | | C: (108/442) | Alzheimer's | Fluoxetine | |
| | | 1.17/3.08E−02 | Disease | | |
| | | Gender | Suicide | | |
| | | Male | Parkinson | | |
| | | C: (99/392) | Huntington's | | |
| | | 1.16/4.50E−02 | Disease | | |
| | | Gender/Dx | Depression | | |
| | | M-PTSD | | | |
| | | C: (10/39) | | | |
| | | 2.19/3.65E−03 | | | |
| | | L: (7/25) | | | |
| | | 3.57/5.36E−03 | | | |
| ZNF24/Zinc Finger Protein 24 | Gender/Dx | All | BP | | 22 |
| | M-PTSD | L: (60/270) | Alzheimer's | | |
| | C: (4/39) | 1.47/8.69E−03 | Disease | | |
| | 0.8/2.59E−02 | Gender | Aggression | | |
| | | Male | Stress | | |
| | | L: (57/239) | | | |
| | | 1.42/1.87E−02 | | | |
| | | Gender/Dx | | | |
| | | M-MDD | | | |
| | | L: (6/46) | | | |
| | | 4.42/1.39E−03 | | | |
| | | M-PTSD | | | |
| | | C: (10/39) | | | |
| | | 2.36/7.01E−04 | | | |
| | | L: (7/25) | | | |
| | | 2.74/2.82E−02 | | | |
| | | M-SZA | | | |
| | | L: (21/47) | | | |
| | | 1.63/3.43E−02 | | | |
| CTNND1 | All | All | Stress | Clozapine | 22 |
| Catenin Delta 1 | C: (22/437) | C: (108/442) | Addiction | | |
| | 0.61/4.76E−02 | 1.29/4.38E−03 | Suicide | | |
| | Gender/Dx | Gender | Alzheimer | | |
| | M-MDD | Female | | | |
| | C: (3/56) | L: (5/33) | | | |
| | 0.81/3.58E−02 | 5.89/7.40E−03 | | | |
| | M-PTSD | Male | | | |
| | C: (4/39) | C: (99/392) | | | |
| | 0.8/2.59E−02 | 1.28/6.59E−03 | | | |
| | | Gender/Dx | | | |
| | | M-BP | | | |
| | | C: (26/128) | | | |
| | | 1.38/3.83E−02 | | | |
| | | M-MDD | | | |
| | | C: (9/78) | | | |
| | | 2.48/8.37E−03 | | | |
| | | M-PTSD | | | |
| | | C: (10/39) | | | |
| | | 2.35/1.04E−02 | | | |

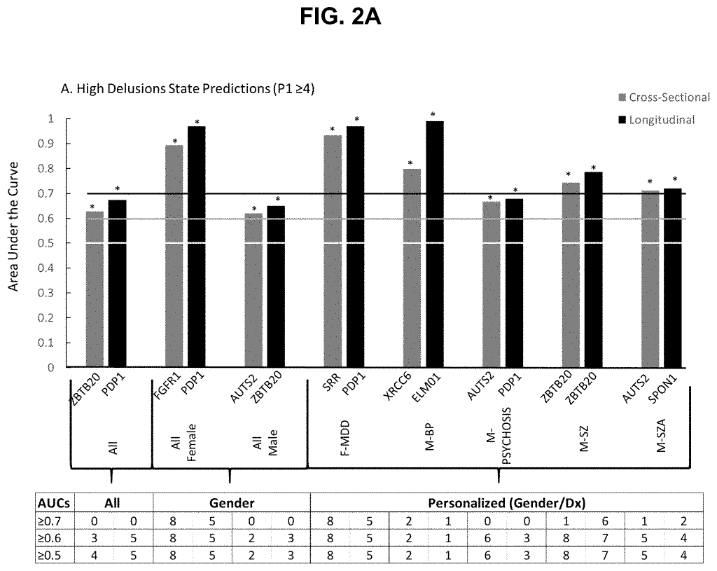

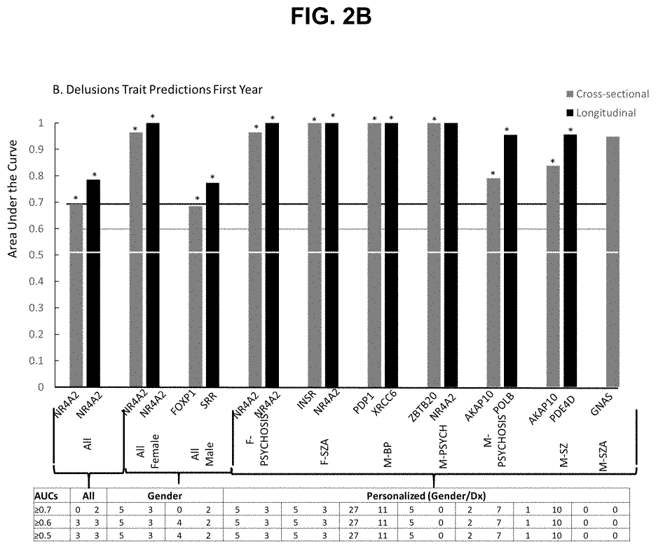

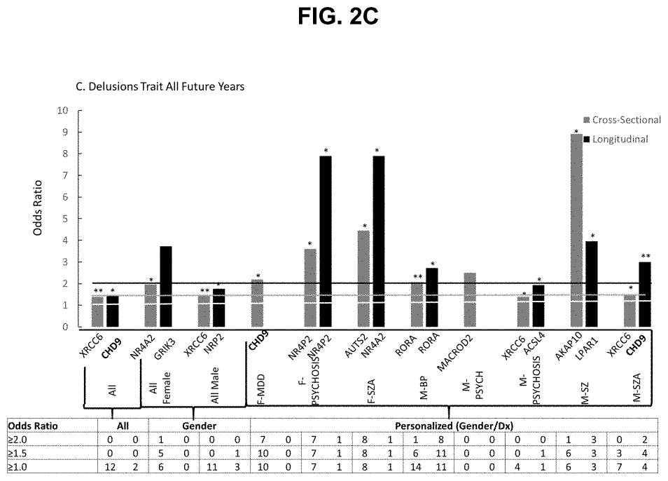

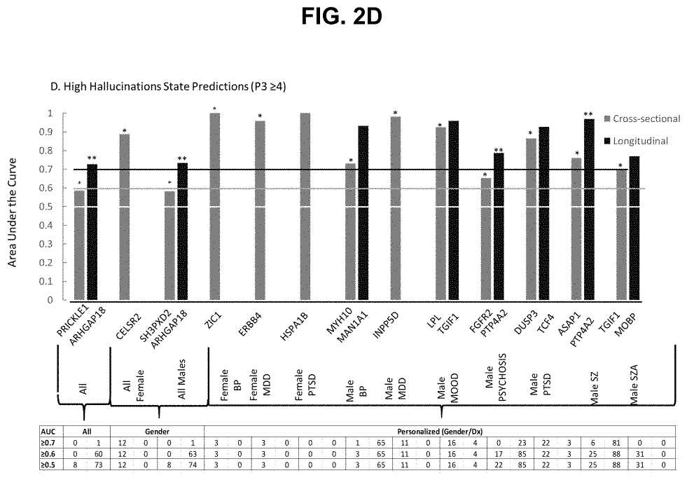

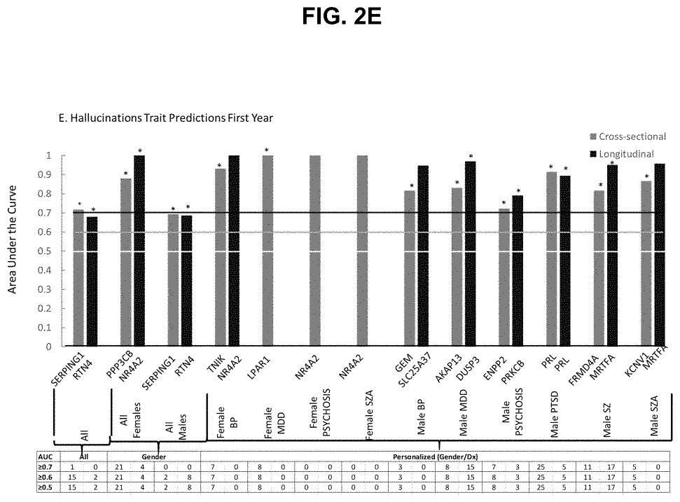

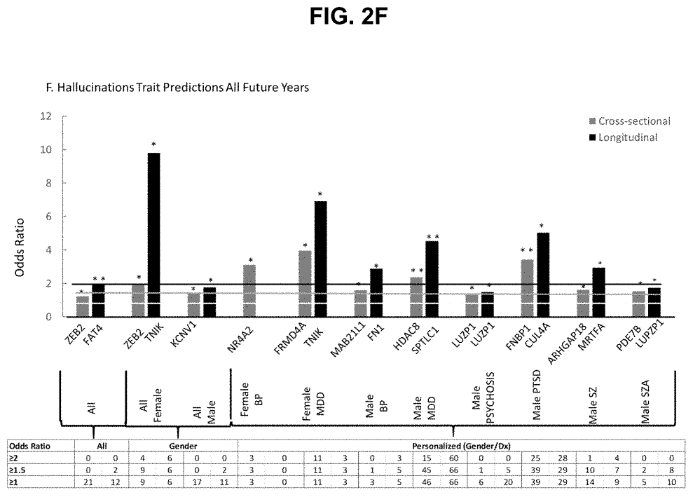

Referring Now to FIG. 2. Top Predictive Biomarkers for Different Demographic and Disease Groups.

Delusions A-C. A. State Severity. B. Short-Term Risk. C. Long-term Risk.

Hallucinations D-F. D. State Severity. E. Short-Term Risk. F. Long-term Risk.

“*”—nominally significant. “**”—significant after Bonferroni correction for number of biomarkers tested. For Delusions, n=70 probesets, 64 genes. For Hallucinations, n=213 probesets, 178 genes.

The number of biomarkers with nominally significant AUCs or Odds Ratios are depicted in the tables underneath the graphs. Bar graph shows best predictive biomarkers in each group. * nominally significant p<0.05. ** survived Bonferroni correction for the number of candidate biomarkers tested. Table underneath the figures displays the actual number of biomarkers for each group whose ROC AUC p-values or Cox Odds Ratio p-values are at least nominally significant. Cross-sectional is based on levels at one visit. Longitudinal is based on levels at multiple visits (integrates levels at most recent visit, maximum levels, slope into most recent visit, and maximum slope). Dividing lines represent the cutoffs for a test performing at chance levels (white), and at the same level as the best biomarkers for all subjects in cross-sectional (gray) and longitudinal (black) based predictions. All depicted biomarkers perform better than chance. Biomarkers performed better when personalized by gender and diagnosis, particularly in females.

Referring now to FIG. 4. Example of Possible Report to Clinicians. Using a panel of the top predictive biomarkers after Step 4. A. Delusions, B. Hallucinations. Each panel contains 18 biomarkers: the top 3 best biomarkers for Current Severity (State) in All, Gender, Gender and Diagnosis; Short-Term Risk (1^{st} Tear) in All, Gender, Gender and Diagnosis; Long-Term Risk (All future) in All, Gender, Gender and Diagnosis.

For the participant and visit for which the report is generated, the raw expression values of the biomarkers were Z-scored by gender and diagnosis with the 793 other participants and visit datasets in our dataset.

The Z-scored expression value of each biomarker in our participant tested was compared to the average value for the biomarker in the 793 dataset, from the severely psychotic group (PANSS item >=4 for state, or having future hospitalizations with psychosis for trait) and from the non-psychotic group (PANSS item=1 for state, or not having future hospitalizations with psychosis for trait.). For increased in expression biomarkers, the comparison resulted in scores of 1 if above the first average, 0 if below the second average, and 0.5 if it was in between. The reverse was done for decreased biomarkers. The comparison groups in the 793 cohort were all, the same gender, and the same gender and diagnosis corresponding to the participant for which the report is generated.

The “digitized” biomarkers were then added into a polygenic risk score, and a percentile calculated. If above the 75%, the patient is deemed high severity/risk (red), if between 75 and 50% is intermediate high, if between 50 and 25% intermediate low, and if below 25% low.

The stars depict each biomarker, and are filled corresponding to the score, and colored corresponding to the level of risk. The “digitized” biomarkers were also used for matching with existing psychiatric medications. Biomarkers were matched based on our CFG literature databases with existing psychiatric medications that had effects on gene expression opposite to psychosis. Each medication matched to a biomarker got a score of one (1) that was then multiplied with the biomarker score of 1, 0.5 or 0. The scores for the medications were added, a percentile calculated, and medications prioritized by this percentile for consideration and use by the clinician.

### A. Delusions.

The participant had a delusions severity score of 63% for current state, 44% for short-term risk, and 11% for long-term risk. This participant's clinical measures were fairly concordant with the blood test results (high delusions scores, poor grooming).

### B. Hallucinations.

The participant had a delusions severity score of 11% for current state, 6% for short-term risk, and 33% for long-term risk. This participant's clinical measures were discordant among themselves and with the blood test results (self-reported high-hallucination scores, but above average grooming), pointing out to the need for objective measures.

TABLE 4
Repurposed Drugs. A-C Delusions. D-F Hallucinations
A. Delusions - All Patients

| | | NIH LINCS Biomarker | |
|---|---|---|---|
| | | Panel Used: | |
| CMAP Biomarker | | Decreased Biomarkers: | |
| Panel Used: | | ZBTB20, FOXP1, | |
| Decreased Biomarkers: | | SPON1, NRP2 | |
| SPON1 | | Increased Biomarkers: | |
| Increased Biomarkers: | | AUTS2, PDP1, NR4A2, | |
| PDP1, NR4A2, IL6ST, | | GNAS, IL6ST, CHD9, XRCC6, | |
| XRCC6, CHD9 | | RORA, ACTN4, ACSL4 | |
| Drug | Score | Drug | Score |
| adenosine phosphate | −1 | 528116.cdx | 0.3077 |
| N-acetyl-L-leucine | −0.974 | Cyclopiazonic Acid | 0.2308 |
| eldeline | −0.96 | SB 218078 | 0.2308 |
| pempidine | −0.958 | BRD-A36630025 | 0.2308 |
| verteporfin | −0.945 | QUINACRINE | 0.2308 |
| | | HYDROCHLORIDE | |
| C-75 | −0.91 | GF-109203X | 0.2308 |
| oxprenolol | −0.909 | BRD-A36630025 | 0.2308 |
| Prestwick-675 | −0.901 | N9- | 0.2308 |
| | | isoproplyolomoucine | |
| meglumine | −0.898 | BMS-536924 | 0.2308 |
| guanethidine | −0.891 | BRD-K76951091 | 0.2308 |
| pancuronium | −0.889 | BRD-K26304855 | 0.2308 |
| bromide | | | |
| karakoline | −0.886 | trichostatin A | 0.2308 |
| 15(S)-15- | −0.885 | ALW-II-38-3 | 0.2308 |
| methylprostaglandin | | | |
| E2 | | | |
| hexylcaine | −0.878 | Mitoxantrone | 0.2308 |
| dicoumarol | −0.878 | HG-6-64-01 | 0.2308 |
| apramycin | −0.878 | Alvocidib | 0.2308 |
| mephenytoin | −0.877 | SB-216763 | 0.2308 |
| estriol | −0.876 | Caffeic acid | 0.1538 |
| | | phenethyl ester | |
| sodium | −0.868 | FR 139317 | 0.1538 |
| phenylbutyrate | | | |
| dienestrol | −0.867 | Syk Inhibitor | 0.1538 |

B. Delusions - Males

| CMAP Biomarker | | NIH LINCS Biomarker | |
|---|---|---|---|
| Panel Used: | | Panel Used: | |
| Decreased Biomarkers: | | Decreased Biomarkers: | |
| ZBTB20, FOXP1, ZBTB20, | | ZBTB20, FOXP1, | |
| SPON1, NRP2 | | SPON1, NRP2 | |
| Increased Biomarkers: | | Increased Biomarkers: | |
| AUTS2, PDP1, FOXP1, | | AUTS2, PDP1, FOXP1, | |
| PDP1, GNAS, SRR, NR4A2, | | GNAS, SRR, NR4A2, | |
| XRCC6, RORA, ACTN4 | | XRCC6, RORA, ACTN4 | |
| Drug | Score | Drug | Score |
| flunisolide | −1 | 528116.cdx | 0.3636 |
| apramycin | −0.995 | SB 218078 | 0.2727 |
| adenosine phosphate | −0.974 | QUINACRINE | 0.2727 |
| | | HYDROCHLORIDE | |
| guanethidine | −0.958 | N9- | 0.2727 |
| | | isoproplyolomoucine | |
| 15(S)-15- | −0.952 | ALW-II-38-3 | 0.2727 |
| methylprostaglandin | | | |
| E2 | | | |
| meteneprost | −0.948 | Mitoxantrone | 0.2727 |
| methyldopate | −0.941 | Mitoxantrone | 0.2727 |
| hydralazine | −0.939 | HG-6-64-01 | 0.2727 |
| rotenone | −0.938 | Alvocidib | 0.2727 |
| phthalylsulfathiazole | −0.936 | SB-216763 | 0.2727 |
| N-acetyl-L-leucine | −0.933 | Syk Inhibitor | 0.1818 |
| eldeline | −0.92 | Cyclopiazonic Acid | 0.1818 |
| tocainide | −0.919 | GW 441756 | 0.1818 |
| laudanosine | −0.918 | LY 225910 | 0.1818 |
| pempidine | −0.918 | AG 82 | 0.1818 |
| 7- | −0.915 | DOXORUBICIN | 0.1818 |
| aminocephalosporanic | | | |
| acid | | | |
| sulfachlorpyridazine | −0.909 | MITOMYCIN C | 0.1818 |
| finasteride | −0.907 | TERFENADINE | 0.1818 |
| verteporfin | −0.905 | DOXORUBICIN | 0.1818 |
| pempidine | −0.904 | Syk Inhibitor | 0.1538 |

C. Delusions - Females

| | |
|---|---|
| CMAP Biomarker | NIH LINCS Biomarker |
| Panel Used: | Panel Used: |
| Decreased Biomarkers: | Decreased Biomarkers: |
| FGFR1, DISC1, FGFR2, | FGFR1, DISC1, FGFR2, |
| SPTBN1, INSR, GRIK3, | SPTBN1, INSR GRIK3, |
| ZBTB20 | ZBTB20 |
| Increased Biomarkers: | Increased Biomarkers: |
| PDE4DIP, PDP1, TCF4, | PDE4DIP, PDP1, TCF4, |
| NR4A2, CHD9, CLCN3, | NR4A2, CHD9, CLCN3, |
| AUTS2, LDB2, NR4A2 | AUTS2, LDB2 |

| Drug | Score | Drug | Score |
|---|---|---|---|
| erastin | −1 | I-BET151 | 0.2667 |
| harpagoside | −0.976 | NYLIDRIN | 0.2 |
| | | HYDROCHLORIDE | |
| metacycline | −0.972 | AMG 9810 | 0.2 |
| amiodarone | −0.969 | DOXORUBICIN | 0.2 |
| furaltadone | −0.958 | MITOMYCIN C | 0.2 |
| metformin | −0.951 | FLUDROCORTISONE | 0.2 |
| | | ACETATE | |
| timolol | −0.935 | Purvalanol A | 0.2 |
| repaglinide | −0.928 | TENIPOSIDE | 0.2 |
| sulfafurazole | −0.92 | Geldanamycin | 0.2 |
| PNU-0230031 | −0.908 | Importazole | 0.2 |
| probenecid | −0.907 | BRD-A36630025 | 0.2 |
| furosemide | −0.904 | YM-155 | 0.2 |
| fluphenazine | −0.896 | Auranofin | 0.2 |
| myricetin | −0.893 | 7643453 | 0.2 |
| sulfacetamide | −0.892 | G-221 | 0.2 |
| lomustine | −0.891 | BRD-A49680073 | 0.2 |
| BCB000039 | −0.889 | BRD-K08547377 | 0.2 |
| harmalol | −0.885 | Cladribine | 0.2 |
| cimetidine | −0.88 | NVP-AUY922 | 0.2 |
| acenocoumarol | −0.877 | TWS-119 | 0.2 |

D. Hallucinations - All Patients

| | | NIH LINCS Biomarker | |
|---|---|---|---|
| | | Panel Used: | |
| | | Decreased Biomarkers: | |
| | | PRL, SERPING1, ENPP2, | |
| CMAP Biomarker | | LAMA4, KCNV1, CTNND1, | |
| Panel Used: | | FAT4 | |
| Decreased Biomarkers: | | Increased Biomarkers: | |
| PRL, SERPING1, ENPP2, | | PRICKLE1, NCAM1, | |
| KCNV1, FAT4 | | B3GALT5, ARHGAP18, | |
| Increased Biomarkers: | | PTP4A2, ACYP2, RTN4, | |
| NCAM1, B3GALT5, PTP4A2, | | CUL4A, ZEB2, DST, | |
| ACYP2, DST | | DLG1 | |
| Drug | Score | Drug | Score |
| clioquinol | −1 | BRD-K71489689 | 0.25 |
| pirinixic acid | −0.949 | trichostatin A | 0.25 |
| moxisylyte | −0.931 | A443654 | 0.25 |
| Prestwick-685 | −0.93 | AG 825 | 0.1875 |
| exemestane | −0.926 | Proscillaridin A | 0.1875 |
| azacitidine | −0.914 | Ala-Ala-Phe-CMK | 0.1875 |
| C-75 | −0.913 | FLUOCINOLONE | 0.1875 |
| | | ACETONIDE | |
| estradiol | −0.894 | manumycin A | 0.1875 |
| tetraethylenepentamine | −0.893 | curcumin | 0.1875 |
| sparteine | −0.887 | BRD-K68548958 | 0.1875 |
| guanethidine | −0.883 | CHR 2797 | 0.1875 |
| idoxuridine | −0.883 | Tyrphostin | 0.1875 |
| | | AG 1478 | |
| gliclazide | −0.878 | wortmannin | 0.1875 |
| nitrendipine | −0.877 | HY-50878 | 0.1875 |
| N-acetyl-L-aspartic | −0.872 | 598226 | 0.1875 |
| acid | | | |
| sulfanilamide | −0.871 | S1003 | 0.1875 |
| doxazosin | −0.87 | BRD-A52530684 | 0.1875 |
| pimozide | −0.865 | CGP-60474 | 0.1875 |
| proscillaridin | −0.864 | buparlisib | 0.1875 |
| oxetacaine | −0.86 | AS-601245 | 0.1875 |

D. Hallucinations - Males

| | NIH LINCS Biomarker |
|---|---|
| | Panel Used: |
| CMAP Biomarker | Decreased Biomarkers: |
| Panel Used: | PRL, SERPING1, ENPP2, |
| Decreased Biomarkers: | KCNV1, MAB21L1, |
| PRL, SERPING1, | CTNND1, FAT4 |
| ENPP2, KCNV1, | Increased Biomarkers: |
| MAB21L1, FAT4 | SH3PXD2A, ZEB2, |
| Increased Biomarkers: | PRICKLE1, ARHGAP18, |
| ACYP2, DST | ACYP2, RTN4, DST |

| Drug | Score | Drug | Score |
|---|---|---|---|
| digoxigenin | −1 | trichostatin A | 0.3333 |
| doxazosin | −0.984 | manumycin A | 0.25 |
| meptazinol | −0.972 | NCGC00189555-02 | 0.25 |
| promethazine | −0.963 | buparlisib | 0.25 |
| cefixime | −0.942 | linifanib | 0.25 |
| velnacrine | −0.929 | AZD-7762 | 0.25 |
| cetirizine | −0.927 | dinaciclib | 0.25 |
| eldeline | −0.924 | Piretanide | 0.1667 |
| atropine oxide | −0.922 | KN-62 | 0.1667 |
| clioquinol | −0.92 | Fluticasone | 0.1667 |
| | | propionate | |
| nicotinic acid | −0.916 | JAK3 Inhibitor VI | 0.1667 |
| clioquinol | −0.915 | SARMENTOGENIN | 0.1667 |
| galantamine | −0.911 | Digoxin | 0.1667 |
| rolitetracycline | −0.909 | MEGESTROL | 0.1667 |
| | | ACETATE | |
| betahistine | −0.903 | Oxymetazoline | 0.1667 |
| | | hydrochloride | |
| sulconazole | −0.9 | U-0126 | 0.1667 |
| monocrotaline | −0.899 | Tracazolate | 0.1667 |
| | | hydrochloride | |
| lanatoside C | −0.895 | FLUFENAMIC | 0.1667 |
| | | ACID | |
| Prestwick-1084 | −0.89 | FENOFIBRATE | 0.1667 |
| naftidrofuryl | −0.887 | U 99194 maleate | 0.1667 |

D. Hallucinations - Females
CMAP Biomarker Panel Used:
Decreased Biomarkers:
GNAS
Increased Biomarkers:
CELSR2, B3GALT5, PPP3CB,
THNSL1, NR4A2

| Drug | Score |
|---|---|
| proglumide | −1 |
| quinethazone | −0.951 |
| esculin | −0.938 |
| MG-262 | −0.935 |
| GW-8510 | −0.932 |
| haloperidol | −0.931 |
| guanethidine | −0.93 |
| deferoxamine | −0.925 |
| citiolone | −0.919 |
| meteneprost | −0.913 |
| amylocaine | −0.91 |
| CP-944629 | −0.907 |
| clemizole | −0.899 |
| IC-86621 | −0.899 |
| nortriptyline | −0.89 |
| CP-944629 | −0.888 |
| tanespimycin | −0.885 |
| Prestwick-674 | −0.883 |
| 0317956-0000 | −0.883 |
| pioglitazone | −0.879 |

### REFERENCES

1. Kurian S M, Le-Niculescu H, Patel S D, et al. Identification of blood biomarkers for psychosis using convergent functional genomics. Mol Psychiatry 2011; 16:37-58.
2. Le-Niculescu H, Levey D F, Ayalew M, et al. Discovery and validation of blood biomarkers for suicidality. Mol Psychiatry 2013; 18:1249-64.
3. Niculescu A B, Levey D F, Phalen P L, et al. Understanding and predicting suicidality using a combined genomic and clinical risk assessment approach. Mol Psychiatry 2015; 20:1266-85.
4. Levey D F, Niculescu E M, Le-Niculescu H, et al. Towards understanding and predicting suicidality in women: biomarkers and clinical risk assessment. Mol Psychiatry 2016; 21:768-85.
5. Niculescu A B, Le-Niculescu H, Levey D F, et al. Precision medicine for suicidality: from universality to subtypes and personalization. Mol Psychiatry 2017; 22:1250-73.

### EMBODIMENTS

Not to be limited to the following embodiments, these embodiments reflect aspects of the methods and uses of the biomarkers identified.

Embodiment [1] A method for assessing and treating schizophrenia and other psychotic disorders in general, in particular delusions and risk of developing delusions in an individual, comprising the steps of:
(a) obtaining a biological sample from an individual and quantifying the amounts of RNA biomarkers in the biological sample, to create a panel of RNA biomarkers,
(b) quantifying the amounts of the RNA biomarkers in the panel in a clinically relevant population to generate a reference expression level for the RNA biomarkers in a panel of RNA biomarkers;
(c) comparing the amounts of the biomarkers in the biological sample from the individual with the amounts of the RNA biomarkers present in the reference standard to generate a score for each biomarker;
wherein the biomarkers in the a first panel (a) comprise one or more of the following RNA biomarkers: Activator Of Transcription and Developmental Regulator 2 (AUTS2), Pyruvate Dehyrogenase Phosphatase Catalytic Subunit 1 (PDP1), Nuclear Receptor Subfamily 4 Group A Member 2 (NR4A2), GNAS Complex Locus (GNAS), Interleukin 6 Signal Transduce (IL6ST), Chromodomain Helicase DNA Binding Protein 9 (CHD9), X-Ray Repair Cross Complementing 6 (XRCC6), RAR Related Orphan Receptor A (RORA), Actinin Alpha 4 (ACTN4), and Acyl-CoA Synthetase Long Chain Family Member 4 (ACSL4), wherein the expression level of the biomarker(s) in the sample is increased relative to a reference expression level, denoting delusions or an increased risk for developing delusions; and
biomarkers in a second panel (b) comprise one or more of the following RNA biomarkers: Zinc Finger And BTB Domain Containing 20 (ZBTB20), Forkhead Box P1 (FOXP1), Spondin 1 (SPON1), and (NRP2), wherein the expression level of the RNA biomarker(s) in the sample is decreased relative to a reference expression level of the RNA biomarkers in the panel, denoting delusions or an increased risk for developing delusions;
(d) generating a score for the panel of RNA biomarkers, based on the scores of the biomarker(s) in the panel;
(e) determining a reference score for the panel in a clinically normal relevant population;
(f) identifying a difference between the score of the panel of biomarker(s) in the sample and the reference score of the panel of biomarker(s);
(g) identifying the individual as having delusions or of having an elevated risk for developing delusions, based on the difference between the biomarker panel score of the individual relative to the biomarker panel score of the reference; and
(h) treating the individual identified as having delusions or an elevated risk of delusions with at least one treatment selected from the group consisting of: a treatment based on clinical practice guidelines, administering a therapeutically effective amount of at least one therapeutic drug wherein the mode of treatment is on the specific biomarkers scores indicating that individual will benefit from a particular therapy.

Embodiment [2] The method of claim [1], wherein the biomarkers are quantified in samples taken on two or more occasions from the individual.

Embodiment [3] The method of claim [1], wherein each biomarker is assigned a weighted coefficient based on each biomarkers importance in in assessing and predicting delusions risk; and the biomarker panel score is based on the weighted coefficients of each of the biomarkers.

Embodiment [4] The method of claim [1], wherein the biological sample is at least sample from the individual selected from the group consisting of: tissue, a fluid such as cerebrospinal fluid, whole blood, blood serum, plasma, saliva, or other bodily fluid, or an extract or purification therefrom, or a dilution thereof.

Embodiment [5]. The method of claim [1], wherein the therapeutic is at least drug selected from the group consisting of: adenosine phosphate, N-acetyl-L-leucine, eldeline, pempidine, verteporfin, C-75, oxprenolol, Prestwick-675, meglumine, guanethidine, pancuronium bromide, karakoline, 15(S)-15-methylprostaglandin E2, hexylcaine, dicoumarol, apramycin, mephenytoin, estriol, 528116.cdx, Cyclopiazonic Acid, SB 218078, BRD-A36630025, Quinacrine hydrochloride, GF-109203X, BRD-A36630025, N9-isoproplyolomoucine, BMS-536924, BRD-K76951091, BRD-K26304855, trichostatin A, ALW-11-38-3, mitoxantrone, HG-6-64-0, alvocidib, SB-216763, and caffeic acid phenethyl ester.

Embodiment [6] The method of claim [1], wherein when the individual is male, and the biomarkers in a first panel (a) comprise one or more biomarkers selected from the group consisting of: Activator Of Transcription And Developmental Regulator (AUTS2), Pyruvate Dehyrogenase Phosphatase Catalytic Subunit 1 (PDP1), Forkhead Box P1 (FOXP1), GNAS Complex Locus (GNAS), Serine Racemase (SRR), Nuclear Receptor Subfamily 4 Group A Member 2 (NR4A2), X-Ray Repair Cross Complementing 6 (XRCC6), RAR Related Orphan Receptor A (RORA), and Actinin Alpha 4 (ACTN4), wherein the expression level of the biomarker(s) in the sample is increased relative to a reference expression level, denoting increased delusions, or

the biomarkers in a second panel (b) comprising one or more biomarkers selected from the group consisting of: Zinc Finger And BTB Domain Containing 20 (ZBTB20), Forkhead Box P1 (FOXP1), Spondin 1 (SPON1), NRP2, wherein the expression level of the biomarker(s) in the sample is decreased relative to a reference expression level, denoting increased delusions.

7. The method of claim 6, wherein the at least one therapeutic drug is one or more drugs selected from the group consisting of: flunisolide, apramycin, adenosine phosphate, guanethidine, 15(S)-15-methylprostaglandin E2, meteneprost, methyldopate, hydralazine, rotenone, phthalylsulfathiazole, N-acetyl-L-leucine, eldeline, tocainide, laudanosine, pempidine, 7-aminocephalosporanic acid, Sulfachlorpyridazine, finasteride, 528116.cdx, SB 218078, Quinacrine hydrochloride, N9-isoproplyolomoucine, ALW-II-38-3, mitoxantrone, HG-6-64-01, Alvocidib, SB-216763, Syk Inhibitor, Cyclopiazonic Acid, GW 441756, LY 225910, AG 82, doxorubicin, mitomycin, and terfenadine.

Embodiment [8] The method of claim [1], wherein when the individual is female, and the biomarkers in the panel comprise one or biomarkers in a first panel (a) comprise one or more of the biomarkers selected from the group consisting of: Phosphodiesterase 4D Interacting Protein (PDE4DIP), Pyruvate Dehyrogenase Phosphatase Catalytic Subunit 1 (PDP1), Transcription Factor 4 (TCF4), Nuclear Receptor Subfamily 4 Group A Member 2 (NR4A2), Chromodomain Helicase DNA Binding Protein 9 (CHD9), (CLCN3), Activator Of Transcription And Developmental Regulator (AUTS2), and (LDB2), wherein the expression level of the biomarker(s) in the sample is increased relative to a reference expression level, denoting increased delusions; and

the biomarkers in a second panel (b) comprise one or more biomarkers selected from the group consisting of (FGFR1), (DISC1), (FGFR2), (SPTBN1), (INSR), (GRIK3), Zinc Finger, and BTB Domain Containing 20 (ZBTB20), wherein the expression level of the biomarker(s) in the sample is decreased relative to a reference expression level, denoting increased delusions.

Embodiment [9] The method of claim [8], wherein the at least one therapeutic drug is at least drug selected from the group consisting of: erastin, harpagoside, metacycline, amiodarone, furaltadone, metformin, timolol, Repaglinide, sulfafurazole, PNU-0230031, Probenecid, furosemide, fluphenazine, myricetin, sulfacetamide, lomustine, BCB000039, Harmalol, I-BET151, Nylidrin hydrochloride, AMG 9810, Doxorubicin, Mitomycin C, Fludrocortisone acetate, Purvalanol A, Teniposide, Geldanamycin, Importazole, BRD-A36630025, YM-155, Auranofin, 7643453, G-221, BRD-A49680073, BRD-K08547377, and Cladribine.

Embodiment [10] A method of assessing and treating schizophrenia and other psychotic disorders in general, and delusions in particular, in an individual, comprising:

calculating combined biomarkers and clinical information Up-based on the equation:

(Biomarker Panel Score)+(Delusions Score)−(Grooming Score)=Up-Delusions Score;

wherein the Biomarker Panel Score is obtained as per the method of claim 1;
wherein the Delusions Score is calculated with a clinical rating or self-report scales;
wherein the Grooming Score is calculated with a rating scale;

assessing the level of delusions of the individual by comparing the individual's Up-Delusions Score to a reference Up-Delusions Score;

administering a treatment for delusions to the individual when the individual's Up-Delusions Score is greater than a reference Up-Suicide Score; and

monitoring the individual's response to a treatment for delusions by determining changes in the Up-Delusions Score after initiating a treatment.

Embodiment [11] A method for assessing and treating schizophrenia and other psychotic disorders in general, in particular hallucinations and risk of developing hallucinations in an individual, comprising the steps of:
(a) obtaining a biological sample from an individual and quantifying the amounts of one or more RNA biomarkers in the biological sample, to create at least one panel of RNA biomarkers,
(b) quantifying the amounts of the RNA biomarkers in the at least one panel in a clinically relevant population to generate a reference expression level for the RNA biomarkers in a panel of RNA biomarkers;
(c) comparing the amounts of the biomarkers in the biological sample from the individual with the amounts of the RNA biomarkers present in the reference standard to generate a score for each biomarker a first panel and a second panel; wherein the biomarkers in the first panel comprise one or more of the following RNA biomarkers: (PRICKLE1), (NCAM1), (B3GALT5), (ARHGAP18), (PTP4A2), Acylphosphatase 2 (ACYP2), Reticulon 4 (RTN4), Cullin 4A (CUL4A), Zinc Finger E-Box Binding Homeobox 2 (ZEB2), Dystonin (DST), and Discs Large MAGUK Scaffold Protein 1 (DLG1), wherein the expression level of the biomarker(s) in the sample is increased relative to a reference expression level, denoting hallucinations or an increased risk for developing hallucinations; and
wherein the biomarkers in the second panel comprise one or more of the following RNA biomarkers: (PRL), (SERPING1), Ectonucleotide Pyrophosphatase/Phosphodiesterase 2 (ENPP2), (LAMA4), (KCNV1), Catenin Delta 1 (CTNND1), and FAT Atypical Cadherin 4 (FAT4), wherein the expression level of the RNA biomarker(s) in the sample is decreased relative to a reference expression level of the RNA biomarkers in the panel, denoting hallucinations or an increased risk for developing hallucinations;
(d) generating a score for the panel of RNA biomarkers, based on the scores of the biomarker(s) in the panel;
(e) determining a reference score for the panel in a clinically normal relevant population;
(f) identifying a difference between the score of the panel of biomarker(s) in the sample and the reference score of the panel of biomarker(s);
(g) identifying the individual as manifesting hallucinations or of having an elevated risk for developing hallucinations, based on the difference between the biomarker panel score of the individual relative to the biomarker panel score of the reference; and
(h) treating the individual identified as having hallucinations or an elevated risk of hallucinations with one or more of the following: 1) a treatment based on clinical practice guidelines, 2) administering a therapeutically effective amount of a therapeutic drug (s), selected based on the specific biomarkers whose scores indicate that they are changed in the individual compared to a reference standard.

Embodiment [12] The method of claim [11], wherein the biomarkers are quantified in samples taken on two or more occasions from the individual.

Embodiment [13] The method of claim [11], wherein each biomarker is assigned a weighted coefficient based on the biomarkers importance in in assessing and predicting hallucinations risk; and

the biomarker panel score is based on the weighted coefficients of each of the biomarkers.

Embodiment [14] The method of claim [11], wherein the biological sample is a tissue sample or a fluid, such as cerebrospinal fluid, whole blood, blood serum, plasma, saliva, or other bodily fluid, or an extract or purification therefrom, or dilution thereof.

Embodiment [15] The method of claim [11], wherein the one or more therapeutic is one or more compounds selected from the group consisting of: clioquinol, pirinixic acid, moxisylyte, Prestwick-685, exemestane, azacytidine, C-75, estradiol, tetraethylenepentamine, sparteine, guanethidine, idoxuridine, gliclazide, nitrendipine, N-acetyl-L-aspartic acid, sulfanilamide, doxazosin, pimozide, Proscillaridin, oxetacaine, BRD-K71489689, trichostatin A, A443654, AG 825, Proscillaridin A, Ala-Ala-Phe-CMK, Fluocinolone acetonide, manumycin A, curcumin, BRD-K68548958, CHR 2797, Tyrphostin AG 1478, Wortmannin, HY-50878, 598226, S1003, BRD-A52530684, CGP-60474, Buparlisib, and AS-601245.

Embodiment [16] The method of claim [11], wherein when the individual is male, and the biomarkers in a first panel (a) comprise one or more biomarkers selected from the group consisting of: (SH3PXD2A), Zinc Finger E-Box Binding Homeobox 2 (ZEB2), (PRICKLE1), (ARHGAP18), Acylphosphatase 2 (ACYP2), Reticulon 4 (RTN4), and Dystonin (DST), wherein the expression level of the biomarker(s) in the sample is increased relative to a reference expression level, denoting increased hallucinations; and

biomarkers in a second panel (b) comprise one or more biomarkers selected from the group consisting of: (PRL), (SERPING1), Ectonucleotide Pyrophosphatase/Phosphodiesterase 2 (ENPP2), (KCNV1), Mab-21 Like 1 (MAB21L1), Catenin Delta 1 (CTNND1), and FAT Atypical Cadherin 4 (FAT4), wherein the expression level of the biomarker(s) in the sample is decreased relative to a reference expression level, denoting increased hallucinations.

Embodiment [17] The method of claim [16], wherein the at least one therapeutic is at least one compound selected from the group consisting of: digoxigenin, doxazosin, meptazinol, promethazine, cefixime, velnacrine, cetirizine, eldeline, atropine oxide, clioquinol, nicotinic acid, clioquinol, galantamine, rolitetracycline, betahistine, sulconazole, monocrotaline, lanatoside C, Prestwick-1084, Naftidrofuryl, sulfachlorpyridazine, helveticoside, bezafibrate, mifepristone, trichostatin A, manumycin A, NCGC00189555-02, Buparlisib, linifanib, AZD-7762, Dinaciclib, Piretanide, KN-62, Fluticasone propionate, JAK3 Inhibitor VI, Sarmentogenin, Digoxin, Megestrol acetate, Oxymetazoline hydrochloride, U-0126, Tracazolate hydrochloride, Flufenamic acid, Fenofibrate, and U 99194 maleate.

Embodiment [18] The method of claim [11], wherein when the individual is female, and the biomarkers in a first panel (a) comprise one or more biomarkers selected from the group consisting of: (CELSR2), (KALRN), (B3GALT5), Protein Phosphatase 3 Catalytic Subunit Beta (PPP3CB), (ZFR), (THNSL1), (TNIK), Nuclear Receptor Subfamily 4 Group A Member 2 (NR4A2), Zinc Finger E-Box Binding Homeobox 2 (ZEB2), and (TNIK), wherein the expression level of the biomarker(s) in the sample is increased relative to a reference expression level, denoting increased hallucinations; and

biomarkers in a second panel (b) comprise one or more biomarkers selected from the group consisting of GNAS Complex Locus (GNAS), and Catenin Delta 1 (CTNND1), wherein the expression level of the biomarker(s) in the sample is decreased relative to a reference expression level, denoting increased hallucinations.

Embodiment [19] The method of claim 18, wherein the at least one therapeutic is at least one compound selected from the group consisting of: proglumide, quinethazone, esculin, MG-262, GW-8510, haloperidol, guanethidine, deferoxamine, citiolone, meteneprost, amylocaine, CP-944629, Clemizole, IC-86621, Nortriptyline, CP-944629, Tanespimycin, Prestwick-674, 0317956-0000, and Pioglitazone.

Embodiment [20] A method of assessing and treating schizophrenia and other psychotic disorders in general, and hallucinations in particular in an individual, comprising:

calculating combined biomarkers and clinical information Up-based on the equation:

(Biomarker Panel Score)+(Hallucinations Score)−(Grooming Score)=Up-Hallucinations Score;
wherein the Biomarker Panel Score is obtained as per the method of claim 11;
wherein the Hallucinations Score is calculated with a clinical rating or self-report scales;
wherein the Grooming Score is calculated with a rating scale;

assessing the level of hallucinations of the individual by comparing the individual's Up-Hallucinations Score to a reference Up-Hallucinations Score;

administering a treatment for hallucinations to the individual when the individual's Up-Hallucinations Score is greater than a reference Up-Suicide Score; and

monitoring the individual's response to a treatment for hallucinations by determining changes in the Up-Hallucinations Score after initiating a treatment.

1. A method for assessing and treating schizophrenia and other psychotic disorders in general, in particular delusions and risk of developing delusions in an individual, comprising the steps of:
(a) obtaining a biological sample from an individual and quantifying the amounts of RNA biomarkers in the biological sample, to create a panel of RNA biomarkers,
(b) quantifying the amounts of the RNA biomarkers in the panel in a clinically relevant population to generate a reference expression level for the RNA biomarkers in a panel of RNA biomarkers;
(c) comparing the amounts of the biomarkers in the biological sample from the individual with the amounts of the RNA biomarkers present in the reference standard to generate a score for each biomarker;
wherein the biomarkers in the a first panel (a) comprise one or more of the following RNA biomarkers: Activator Of Transcription and Developmental Regulator 2 (AUTS2), Pyruvate Dehyrogenase Phosphatase Catalytic Subunit 1 (PDP1), Nuclear Receptor Subfamily 4 Group A Member 2 (NR4A2), GNAS Complex Locus (GNAS), Interleukin 6 Signal Transduce (IL6ST), Chromodomain Helicase DNA Binding Protein 9 (CHD9), X-Ray Repair Cross Complementing 6 (XRCC6), RAR Related Orphan Receptor A (RORA), Actinin Alpha 4 (ACTN4), and Acyl-CoA Synthetase Long Chain Family Member 4 (ACSL4), wherein the expression level of the biomarker(s) in the sample is increased relative to a reference expression level, denoting delusions or an increased risk for developing delusions; and
biomarkers in a second panel (b) comprise one or more of the following RNA biomarkers: Zinc Finger And BTB Domain Containing 20 (ZBTB20), Forkhead Box P1 (FOXP1), Spondin 1 (SPON1), and (NRP2), wherein the expression level of the RNA biomarker(s) in the sample is decreased relative to a reference expression level of the RNA biomarkers in the panel, denoting delusions or an increased risk for developing delusions;
(d) generating a score for the panel of RNA biomarkers, based on the scores of the biomarker(s) in the panel;
(e) determining a reference score for the panel in a clinically normal relevant population;
(f) identifying a difference between the score of the panel of biomarker(s) in the sample and the reference score of the panel of biomarker(s);
(g) identifying the individual as having delusions or of having an elevated risk for developing delusions, based on the difference between the biomarker panel score of the individual relative to the biomarker panel score of the reference;
(h) treating the individual identified as having delusions or an elevated risk of delusions with at least one treatment selected from the group consisting of: a treatment based on clinical practice guidelines, administering a therapeutically effective amount of at least one therapeutic drug wherein the mode of treatment is on the specific biomarkers scores indicating that individual will benefit from a particular therapy.

2. The method of claim 1, wherein the biomarkers are quantified in samples taken on two or more occasions from the individual.

3. The method of claim 1, wherein each biomarker is assigned a weighted coefficient based on each biomarkers importance in in assessing and predicting delusions risk; and the biomarker panel score is based on the weighted coefficients of each of the biomarkers.

4. The method of claim 1, wherein the biological sample is at least sample from the individual selected from the group consisting of: tissue, a fluid such as cerebrospinal fluid, whole blood, blood serum, plasma, saliva, or other bodily fluid, or an extract or purification therefrom, or a dilution thereof.

5. The method of claim 1, wherein the therapeutic is at least drug selected from the group consisting of: adenosine phosphate, N-acetyl-L-leucine, eldeline, pempidine, verteporfin, C-75, oxprenolol, Prestwick-675, meglumine, guanethidine, pancuronium bromide, karakoline, 15(S)-15-methylprostaglandin E2, hexylcaine, dicoumarol, apramycin, mephenytoin, estriol, 528116.cdx, Cyclopiazonic Acid, SB 218078, BRD-A36630025, Quinacrine hydrochloride, GF-109203X, BRD-A36630025, N9-isoproplyolomoucine, BMS-536924, BRD-K76951091, BRD-K26304855, trichostatin A, ALW-II-38-3, mitoxantrone, HG-6-64-0, alvocidib, SB-216763, and caffeic acid phenethyl ester.

6. The method of claim 1, wherein when the individual is male, and the biomarkers in a first panel (a) comprise one or more biomarkers selected from the group consisting of: Activator Of Transcription And Developmental Regulator (AUTS2), Pyruvate Dehyrogenase Phosphatase Catalytic Subunit 1 (PDP1), Forkhead Box P1 (FOXP1), GNAS Complex Locus (GNAS), Serine Racemase (SRR), Nuclear Receptor Subfamily 4 Group A Member 2 (NR4A2), X-Ray Repair Cross Complementing 6 (XRCC6), RAR Related Orphan Receptor A (RORA), and Actinin Alpha 4 (ACTN4), wherein the expression level of the biomarker(s) in the sample is increased relative to a reference expression level, denoting increased delusions, or
the biomarkers in a second panel (b) comprising one or more biomarkers selected from the group consisting of: Zinc Finger And BTB Domain Containing 20 (ZBTB20), Forkhead Box P1 (FOXP1), Spondin 1 (SPON1), NRP2, wherein the expression level of the biomarker(s) in the sample is decreased relative to a reference expression level, denoting increased delusions.

7. The method of claim 6, wherein the at least one therapeutic drug is one or more drugs selected from the group consisting of: flunisolide, apramycin, adenosine phosphate, guanethidine, 15(S)-15-methylprostaglandin E2, meteneprost, methyldopate, hydralazine, rotenone, phthalylsulfathiazole, N-acetyl-L-leucine, eldeline, tocainide, laudanosine, pempidine, 7-aminocephalosporanic acid, Sulfachlorpyridazine, finasteride, 528116.cdx, SB 218078, Quinacrine hydrochloride, N9-isoproplyolomoucine, ALW-II-38-3, mitoxantrone, HG-6-64-01, Alvocidib, SB-216763, Syk Inhibitor, Cyclopiazonic Acid, GW 441756, LY 225910, AG 82, doxorubicin, mitomycin, and terfenadine.

8. The method of claim 1, wherein when the individual is female, and the biomarkers in the panel comprise one or biomarkers in a first panel (a) comprise one or more of the biomarkers selected from the group consisting of: Phosphodiesterase 4D Interacting Protein (PDE4DIP), Pyruvate Dehyrogenase Phosphatase Catalytic Subunit 1 (PDP1), Transcription Factor 4 (TCF4), Nuclear Receptor Subfamily 4 Group A Member 2 (NR4A2), Chromodomain Helicase DNA Binding Protein 9 (CHD9), (CLCN3), Activator Of Transcription And Developmental Regulator (AUTS2), and (LDB2), wherein the expression level of the biomarker(s) in the sample is increased relative to a reference expression level, denoting increased delusions; and
the biomarkers in a second panel (b) comprise one or more biomarkers selected from the group consisting of (FGFR1), (DISC1), (FGFR2), (SPTBN1), (INSR), (GRIK3), Zinc Finger, and BTB Domain Containing 20 (ZBTB20), wherein the expression level of the biomarker(s) in the sample is decreased relative to a reference expression level, denoting increased delusions.

9. The method of claim 8, wherein the at least one therapeutic drug is at least drug selected from the group consisting of: erastin, harpagoside, metacycline, amiodarone, furaltadone, metformin, timolol, Repaglinide, sulfafurazole, PNU-0230031, Probenecid, furosemide, fluphenazine, myricetin, sulfacetamide, lomustine, BCB000039, Harmalol, I-BET151, Nylidrin hydrochloride, AMG 9810, Doxorubicin, Mitomycin C, Fludrocortisone acetate, Purvalanol A, Teniposide, Geldanamycin, Importazole, BRD-A36630025, YM-155, Auranofin, 7643453, G-221, BRD-A49680073, BRD-K08547377, and Cladribine.

10. A method of assessing and treating schizophrenia and other psychotic disorders in general, and delusions in particular, in an individual, comprising:
calculating combined biomarkers and clinical information Up-based on the equation:
(Biomarker Panel Score)+(Delusions Score)−(Grooming Score)=Up-Delusions Score;
wherein the Biomarker Panel Score is obtained as per the method of claim 1;
wherein the Delusions Score is calculated with a clinical rating or self-report scales;
wherein the Grooming Score is calculated with a rating scale;
assessing the level of delusions of the individual by comparing the individual's Up-Delusions Score to a reference Up-Delusions Score;
administering a treatment for delusions to the individual when the individual's Up-Delusions Score is greater than a reference Up-Suicide Score; and
monitoring the individual's response to a treatment for delusions by determining changes in the Up-Delusions Score after initiating a treatment.

11. A method for assessing and treating schizophrenia and other psychotic disorders in general, in particular hallucinations and risk of developing hallucinations in an individual, comprising the steps of:
(a) obtaining a biological sample from an individual and quantifying the amounts of one or more RNA biomarkers in the biological sample, to create at least one panel of RNA biomarkers,
(b) quantifying the amounts of the RNA biomarkers in the at least one panel in a clinically relevant population to generate a reference expression level for the RNA biomarkers in a panel of RNA biomarkers;
(c) comparing the amounts of the biomarkers in the biological sample from the individual with the amounts of the RNA biomarkers present in the reference standard to generate a score for each biomarker a first panel and a second panel; wherein the biomarkers in the first panel comprise one or more of the following RNA biomarkers: (PRICKLE1), (NCAM1), (B3GALT5), (ARHGAP18), (PTP4A2), Acylphosphatase 2 (ACYP2), Reticulon 4 (RTN4), Cullin 4A (CUL4A), Zinc Finger E-Box Binding Homeobox 2 (ZEB2), Dystonin (DST), and Discs Large MAGUK Scaffold Protein 1 (DLG1), wherein the expression level of the biomarker(s) in the sample is increased relative to a reference expression level, denoting hallucinations or an increased risk for developing hallucinations; and
wherein the biomarkers in the second panel comprise one or more of the following RNA biomarkers: (PRL), (SERPING1), Ectonucleotide Pyrophosphatase/Phosphodiesterase 2 (ENPP2), (LAMA4), (KCNV1), Catenin Delta 1 (CTNND1), and FAT Atypical Cadherin 4 (FAT4), wherein the expression level of the RNA biomarker(s) in the sample is decreased relative to a reference expression level of the RNA biomarkers in the panel, denoting hallucinations or an increased risk for developing hallucinations;
(d) generating a score for the panel of RNA biomarkers, based on the scores of the biomarker(s) in the panel;
(e) determining a reference score for the panel in a clinically normal relevant population;
(f) identifying a difference between the score of the panel of biomarker(s) in the sample and the reference score of the panel of biomarker(s);
(g) identifying the individual as manifesting hallucinations or of having an elevated risk for developing hallucinations, based on the difference between the biomarker panel score of the individual relative to the biomarker panel score of the reference;
(h) treating the individual identified as having hallucinations or an elevated risk of hallucinations with one or more of the following: 1) a treatment based on clinical practice guidelines, 2) administering a therapeutically effective amount of a therapeutic drug (s), selected based on the specific biomarkers whose scores indicate that they are changed in the individual compared to a reference standard.

12. The method of claim 11, wherein the biomarkers are quantified in samples taken on two or more occasions from the individual.

13. The method of claim 11, wherein each biomarker is assigned a weighted coefficient based on the biomarkers importance in in assessing and predicting hallucinations risk; and the biomarker panel score is based on the weighted coefficients of each of the biomarkers.

14. The method of claim 11, wherein the biological sample is a tissue sample or a fluid, such as cerebrospinal fluid, whole blood, blood serum, plasma, saliva, or other bodily fluid, or an extract or purification therefrom, or dilution thereof.

15. The method of claim 11, wherein the one or more therapeutic is one or more compounds selected from the group consisting of: clioquinol, pirinixic acid, moxisylyte, Prestwick-685, exemestane, azacytidine, C-75, estradiol, tetraethylenepentamine, sparteine, guanethidine, idoxuridine, gliclazide, nitrendipine, N-acetyl-L-aspartic acid, sulfanilamide, doxazosin, pimozide, Proscillaridin, oxetacaine, BRD-K71489689, trichostatin A, A443654, AG 825, Proscillaridin A, Ala-Ala-Phe-CMK, Fluocinolone acetonide, manumycin A, curcumin, BRD-K68548958, CHR 2797, Tyrphostin AG 1478, Wortmannin, HY-50878, 598226, S1003, BRD-A52530684, CGP-60474, Buparlisib, and AS-601245.

16. The method of claim 11, wherein when the individual is male, and the biomarkers in a first panel (a) comprise one or more biomarkers selected from the group consisting of:
(SH3PXD2A), Zinc Finger E-Box Binding Homeobox 2 (ZEB2), (PRICKLE1), (ARHGAP18), Acylphosphatase 2 (ACYP2), Reticulon 4 (RTN4), and Dystonin (DST), wherein the expression level of the biomarker(s) in the sample is increased relative to a reference expression level, denoting increased hallucinations; and
biomarkers in a second panel (b) comprise one or more biomarkers selected from the group consisting of: (PRL), (SERPING1), Ectonucleotide Pyrophosphatase/Phosphodiesterase 2 (ENPP2), (KCNV1), Mab-21 Like 1 (MAB21L1), Catenin Delta 1 (CTNND1), and FAT Atypical Cadherin 4 (FAT4), wherein the expression level of the biomarker(s) in the sample is decreased relative to a reference expression level, denoting increased hallucinations.

17. The method of claim 16, wherein the at least one therapeutic is at least one compound selected from the group consisting of: digoxigenin, doxazosin, meptazinol, promethazine, cefixime, velnacrine, cetirizine, eldeline, atropine oxide, clioquinol, nicotinic acid, clioquinol, galantamine, rolitetracycline, betahistine, sulconazole, monocrotaline, lanatoside C, Prestwick-1084, Naftidrofuryl, sulfachlorpyridazine, helveticoside, bezafibrate, mifepristone, trichostatin A, manumycin A, NCGC00189555-02, Buparlisib, linifanib, AZD-7762, Dinaciclib, Piretanide, KN-62, Fluticasone propionate, JAK3 Inhibitor VI, Sarmentogenin, Digoxin, Megestrol acetate, Oxymetazoline hydrochloride, U-0126, Tracazolate hydrochloride, Flufenamic acid, Fenofibrate, and U 99194 maleate.

18. The method of claim 11, wherein when the individual is female, and the biomarkers in a first panel (a) comprise one or more biomarkers selected from the group consisting of:
(CELSR2), (KALRN), (B3GALT5), Protein Phosphatase 3 Catalytic Subunit Beta (PPP3CB), (ZFR), (THNSL1), (TNIK), Nuclear Receptor Subfamily 4 Group A Member 2 (NR4A2), Zinc Finger E-Box Binding Homeobox 2 (ZEB2), and (TNIK), wherein the expression level of the biomarker(s) in the sample is increased relative to a reference expression level, denoting increased hallucinations; and
biomarkers in a second panel (b) comprise one or more biomarkers selected from the group consisting of GNAS Complex Locus (GNAS), and Catenin Delta 1 (CTNND1), wherein the expression level of the biomarker(s) in the sample is decreased relative to a reference expression level, denoting increased hallucinations.

19. The method of claim 18, wherein the at least one therapeutic is at least one compound selected from the group consisting of: proglumide, quinethazone, esculin, MG-262, GW-8510, haloperidol, guanethidine, deferoxamine, citiolone, meteneprost, amylocaine, CP-944629, Clemizole, IC-86621, Nortriptyline, CP-944629, Tanespimycin, Prestwick-674, 0317956-0000, and Pioglitazone.

20. A method of assessing and treating schizophrenia and other psychotic disorders in general, and hallucinations in particular in an individual, comprising:
calculating combined biomarkers and clinical information Up-based on the equation:
(Biomarker Panel Score)+(Hallucinations Score)−(Grooming Score)=Up-Hallucinations Score;
wherein the Biomarker Panel Score is obtained as per the method of claim 11;
wherein the Hallucinations Score is calculated with a clinical rating or self-report scales;
wherein the Grooming Score is calculated with a rating scale;
assessing the level of hallucinations of the individual by comparing the individual's Up-Hallucinations Score to a reference Up-Hallucinations Score;
administering a treatment for hallucinations to the individual when the individual's Up-Hallucinations Score is greater than a reference Up-Suicide Score; and
monitoring the individual's response to a treatment for hallucinations by determining changes in the Up-Hallucinations Score after initiating a treatment.
